(19) United States
(12) Patent Application Publication
(10) Pub. No.: US 2019/0016984 A1
(43) Pub. Date: Jan. 17, 2019

(54) CONTINUOUS PROCESS FOR THE MANUFACTURE OF GREASE

(71) Applicant: ExxonMobil Research and Engineering Company, Annandale, NJ (US)

(72) Inventors: Craig J. EMETT, Houston, TX (US); John K. GRAHAM, Ardmore, PA (US); John ZACHARKOW, Burlington, NJ (US); Nabila BRABEZ, Logan Township, NJ (US); James E. SPAGNOLI, Leland, NC (US)

(21) Appl. No.: 16/029,873

(22) Filed: Jul. 9, 2018

Related U.S. Application Data
(60) Provisional application No. 62/532,048, filed on Jul. 13, 2017.

(51) Int. Cl. C10M 117/04; C10M 169/02; C10M 177/00; C07C 51/41; C07C 51/44

(52) U.S. Cl. CPC C10M 117/04; C10M 169/02; C10M 177/00; C07C 51/412; C10N 2270/00; C10M 2203/003; C10M 2207/1285; C10N 2250/10; C07C 51/44

(57) ABSTRACT

This disclosure relates to a continuous process for preparing a grease thickener product in which operating conditions of the continuous process can be varied to obtain desired grease product properties. The process includes: (a) introducing a C₃ to C₃₀ hydroxy fatty acid or a C₃ to C₃₀ hydroxy fatty acid ester, and a metal base into a reaction zone; (b) operating the reaction zone at a temperature between 100° C. and 240° C. to react the C₃ to C₃₀ hydroxy fatty acid or the C₃ to C₃₀ hydroxy fatty acid ester, and the metal base, to produce a grease thickener product; (c) introducing the grease thickener product of step (b) into a flash chamber zone; and (d) operating the flash chamber zone at a temperature sufficient to remove volatiles from the grease thickener product of step (b). The continuous process conditions afford a production throughput of grease thickener product of greater than 8,000 pounds per hour.

### CROSS-REFERENCE TO RELATED APPLICATIONS

This application claims the benefit of U.S. Provisional Application No. 62/532,048, filed on Jul. 13, 2017, the entire contents of which are incorporated herein by reference.

### FIELD

This disclosure relates to a continuous process for manufacturing soap thickened grease compositions in which operating conditions of the continuous process can be varied to obtain desired grease product properties. In particular, the continuous process affords opportunities to specify a combination of formulation components and operating parameters (e.g., flow rates, recycle rates, temperatures, pressures, etc.) that enable the manufacture of greases with very different characteristics that provide optimum performance in a wide variety of diverse industrial and automotive applications.

### BACKGROUND

Grease production in the industry is generally performed in large kettles and contactors. During these batch productions, raw materials must be heated and cooled over long periods of time. Turnaround time for each batch generally exceeds 10 hours, and raw materials are added manually by an operator.

In grease production, lithium soaps are used and are classified into two types, namely, simple soaps and complex soaps. The main performance difference between the two is the greater thermal stability of the lithium complex greases. Both types contain the lithium salt of a fatty acid. Lithium complex soaps contain an additional lithium salt, or “complexing agent”, most commonly a dilithium salt of a low molecular weight dibasic organic acid or dibasic ester.

In addition to batch productions, lithium greases can be manufactured by continuous processes, for example, in a heated tube reactor. The process is most convenient and problem-free for simple lithium soap thickened greases. However, lithium complex greases having much higher thickener concentrations experience increased difficulties during continuous production. These difficulties include reduced unit production capacity including wide fluctuations in flow rates and cessation of flow and resultant downtime.

Continuous production of grease can allow for manufacture at much higher rates than batch units of similar scale. For example, a continuous manufacturing unit can produce grease at rates over 12,000 pounds per hour. Conversely, a typical large grease kettle produces 40,000 lbs of grease in 12 hours, equating to just over 3,000 pounds per hour production rate.

Because the grease demand continues to increase, a more efficient production process is needed to produce large quantities of grease, including both simple and complex lithium soap thickened greases. A continuous process is needed for faster production of grease, in particular, a process that advantageously drives more efficient deployment of capital due to economies of scale, and pushes down manufacturing cost per product unit.

### SUMMARY

This disclosure relates in part to an efficient and fast process for the production large quantities of grease, including both simple and complex lithium soap thickened greases. The continuous process of this disclosure advantageously drives more efficient deployment of capital due to economies of scale, and pushes down manufacturing cost per product unit.

This disclosure also relates in part to a continuous process for preparing a grease thickener product. The process comprises: (a) introducing a C₃ to C₃₀ hydroxy fatty acid or a C₃ to C₃₀ hydroxy fatty acid ester, and a metal base into a reaction zone; (b) operating the reaction zone at a temperature between about 100° C. and about 240° C. to react the C₃ to C₃₀ hydroxy fatty acid or the C₃ to C₃₀ hydroxy fatty acid ester, and the metal base, to produce a grease thickener product; (c) introducing the grease thickener product of step (b) into a flash chamber zone; and (d) operating the flash chamber zone at a temperature sufficient to remove volatiles from the grease thickener product of step (b). The continuous process conditions afford a production throughput of grease thickener product of greater than about 8,000 pounds per hour, preferably greater than about 10,000 pounds per hour, and more preferably greater than about 12,000 pounds per hour.

This disclosure further relates in part to a continuous process for preparing a grease reaction product. The process comprises: (a) passing one or more base oils through a heat exchanger operated at a temperature between about 100° C. and 240° C.; (b) introducing the one or more base oils of step (a), a C₃ to C₃₀ hydroxy fatty acid or a C₃ to C₃₀ hydroxy fatty acid ester, and a metal base into a reaction zone; (c) operating the reaction zone at a temperature between about 100° C. and about 240° C. to react the C₃ to C₃₀ hydroxy fatty acid or the C₃ to C₃₀ hydroxy fatty acid ester, and the metal base, in the presence of the one or more base oils, to produce a grease reaction product; (d) introducing the grease reaction product of step (c) into a flash chamber zone; and (e) operating the flash chamber zone at a temperature sufficient to remove volatiles from the grease reaction product of step (c). The continuous process conditions afford a production throughput of grease reaction product of greater than about 8,000 pounds per hour, preferably greater than about 10,000 pounds per hour, and more preferably greater than about 12,000 pounds per hour.

This disclosure yet further relates in part to a continuous process for preparing a grease thickener product. The process comprises: conducting a neutralization reaction comprising reacting a C₃ to C₃₀ hydroxy fatty acid or a C₃ to C₃₀ hydroxy fatty acid ester, and a metal base, under neutralization reaction conditions sufficient to produce a grease thickener product comprising a metal salt of the C₃ to C₃₀ hydroxy fatty acid or the C₃ to C₃₀ hydroxy fatty acid ester; conducting a nucleation reaction under nucleation reaction conditions sufficient for the metal salt of the C₃ to C₃₀ hydroxy fatty acid or the C₃ to C₃₀ hydroxy fatty acid ester to self-assemble forming a fibrillary network; and conducting a phase separation under phase separation conditions sufficient to remove byproducts from the grease thickener product, said byproducts comprising methanol and water. The neutralization reaction is conducted at a temperature above the melting temperature of the C₃ to C₃₀ hydroxy fatty acid or the C₃ to C₃₀ hydroxy fatty acid ester and below the melting temperature of the metal salt of the C₃ to C₃₀ hydroxy fatty acid or the C₃ to C₃₀ hydroxy fatty acid ester. As the metal salt of the C₃ to C₃₀ hydroxy fatty acid or the C₃ to C₃₀ hydroxy fatty acid ester is produced in the neutralization reaction, the difference between the melting temperature of the metal salt of the C₃ to C₃₀ hydroxy fatty acid or the C₃ to C₃₀ hydroxy fatty acid ester and the melting temperature of the C₃ to C₃₀ hydroxy fatty acid or the C₃ to C₃₀ hydroxy fatty acid ester creates a chemical driving force that initiates the nucleation reaction forming the fibrillary network.

This disclosure relates in part to a continuous process for preparing a grease reaction product. The process comprises: conducting a neutralization reaction comprising reacting a C₃ to C₃₀ hydroxy fatty acid or a C₃ to C₃₀ hydroxy fatty acid ester, and a metal base, in the presence of one or more base oils, under neutralization reaction conditions sufficient to produce a grease reaction product comprising a metal salt of the C₃ to C₃₀ hydroxy fatty acid or the C₃ to C₃₀ hydroxy fatty acid ester; conducting a nucleation reaction under nucleation reaction conditions sufficient for the metal salt of the C₃ to C₃₀ hydroxy fatty acid or the C₃ to C₃₀ hydroxy fatty acid ester to self-assemble forming a fibrillary network; and conducting a phase separation under phase separation conditions sufficient to remove byproducts from the grease reaction product, said byproducts comprising methanol and water. The neutralization reaction is conducted at a temperature above the melting temperature of the C₃ to C₃₀ hydroxy fatty acid or the C₃ to C₃₀ hydroxy fatty acid ester and below the melting temperature of the metal salt of the C₃ to C₃₀ hydroxy fatty acid or the C₃ to C₃₀ hydroxy fatty acid ester. As the metal salt of the C₃ to C₃₀ hydroxy fatty acid or the C₃ to C₃₀ hydroxy fatty acid ester is produced in the neutralization reaction, the difference between the melting temperature of the metal salt of the C₃ to C₃₀ hydroxy fatty acid or the C₃ to C₃₀ hydroxy fatty acid ester and the melting temperature of the C₃ to C₃₀ hydroxy fatty acid or the C₃ to C₃₀ hydroxy fatty acid ester creates a chemical driving force that initiates the nucleation reaction forming the fibrillary network.

This disclosure also relates in part to grease thickener products and grease reaction products prepared by the continuous processes of this disclosure.

It has been surprisingly found that, in accordance with this disclosure, large quantities of grease, including both simple and complex lithium soap thickened greases, can be produced in an efficient and fast continuous process. Selection of appropriate continuous process conditions can afford a production throughput of grease product of greater than about 8,000 pounds per hour, preferably greater than about 10,000 pounds per hour, and more preferably greater than about 12,000 pounds per hour. The continuous process of this disclosure advantageously drives more efficient deployment of capital due to economies of scale, and pushes down manufacturing cost per product unit.

Also, it has been surprisingly found that, in accordance with this disclosure, operating conditions of the continuous process can be varied to obtain desired grease product properties. In particular, it has been surprisingly found that, in accordance with this disclosure, the continuous process affords opportunities to specify a combination of formulation components and operating parameters (e.g., flow rates, recycle rates, temperatures, pressures, etc.) that enable the manufacture of greases with very different characteristics that provide optimum performance in a wide variety of diverse industrial and automotive applications. For example: electric motors, automotive wheel bearings, paper machine roll bearings (wet and dry), and wind turbines require different degrees of structural stability and oil release rates, responding to mechanical and thermal stress. These properties are determined mostly by the thickener characteristics of the grease, which is controlled by the operating condition of an in-line grease unit (ILGU).

Other objects and advantages of the present disclosure will become apparent from the detailed description that follows.

### BRIEF DESCRIPTION OF THE DRAWINGS

FIG. 1 shows a general flow diagram of an illustrative ILGU of this disclosure.

FIG. 2 is a schematic representation of a flash chamber used in the ILGU of this disclosure.

### DETAILED DESCRIPTION

All numerical values within the detailed description and the claims herein are modified by “about” or “approximately” the indicated value, and take into account experimental error and variations that would be expected by a person having ordinary skill in the art.

The continuous grease process of the disclosure comprises a saponification including neutralization and nucleation, and phase separation steps carried out in a continuous manner with recycling of the grease mixture through a reactor recycle pump during the saponification step, and recycling the grease mixture through a flash chamber recycle valve and a flash chamber recycle pump during the phase separation step. In an embodiment of the disclosure, the saponification step is carried out in such a manner that recirculating or mixing is maintained during the reaction. The process also comprises a finishing step, which may be carried out by the addition of base oil at a lower temperature than the grease mixture and by passing the grease mixture through a finishing or shear valve.

The apparatus which can be used to prepare the lithium simple and complex soap greases according to the process of the instant disclosure is described in U.S. Pat. Nos. 1,046,090, 3,475,335, 3,475,337, 4,297,227, 4,435,299, 4,444,669, 4,483,776, 4,582,619, and 5,476,600, all of which are hereby incorporated by reference.

In particular, the apparatus for carrying out this continuous grease making process comprises a reaction zone or saponification zone that can be a flow type reactor, and provided with a recycle system for recycling the saponification mixture through the reactor at a sufficient rate to maintain flow. The apparatus also comprises a flash chamber zone for the volatiles removal and soap conditioning steps, provided with a recycle line containing a flash chamber recycle valve and a flash chamber recycle pump for recycling the grease mixture, together with a finishing or shear valve. The apparatus preferably comprises a finishing section with provisions for cooling the grease mixture either by the addition of cold base oil or by passing it through a finishing or shear valve.

In carrying out the process under the preferred conditions, the saponification zone is maintained at an elevated temperature and pressure at least sufficient to maintain the water and methanol present in the saponification mixture in the liquid phase, and the flash chamber zone is operated at an elevated temperature below the melting point of the soap and under a substantially lower pressure than the saponification zone, so that the major portion of the water and methanol is flashed off when the grease mixture enters the flash chamber zone. Any remaining water and methanol is removed during recycling of the grease mixture through the flash chamber recycle valve, which in effect subjects the grease mixture to a continuous flashing operation by pressure release of the recycle stream through the valve. The flash chamber recycling is preferably carried out at a rapid rate, such that the grease mixture is subjected to multiple passes through the flash chamber recycle valve operated with at least a substantial pressure drop during the residence time of the grease mixture within the flash chamber zone. Cooling of the grease mixture is preferably carried out with the addition of base oil at a substantially lower temperature than the grease mixture, and advantageously in some cases with recycling of the grease mixture through a cooler.

Greases of excellent quality are obtained in the above manner in good yields and in greatly reduced manufacturing times as compared with the prior art processes.

In an embodiment, the continuous grease manufacturing process reacts a saponifiable material with an aqueous solution of a metal base in a saponification zone at a saponification reaction temperature and superatmospheric pressure to produce a saponification reaction product comprising soap. The saponification reaction temperature is below the soap melting temperature. The saponification reaction product passes through a pressure reducing means to effect pressure reduction to flash vaporize substantially all the water and methanol present in the saponification reaction product. The saponification reaction product then passes through a heat exchanger to heat the saponification reaction product to a dehydration temperature. The saponification reaction product then passes to a flash chamber or dehydration zone to produce a dehydrated saponification reaction product. A first portion of the dehydrated saponification reaction product recycles through a shear valve for conditioning the soap contained therein. Then base oil is added to the recycled dehydrated saponification reaction product to produce a grease product.

A more specific continuous grease manufacturing process for preparing a lithium simple or complex soap grease is carried out as follows. A mixture of a C₁₂ to C₂₄ hydroxy fatty acid, a lithium base, and a lubricating oil are continuously introduced into a reaction zone. The lubricating oil (i.e., base oil) employed in making these greases may be any suitable oil having lubricating characteristics, including both conventional mineral oils and synthetic oils or blends thereof. This includes API Group I, II, III, IV, and V and blends having viscosities of between 5 cSt to 3200 cSt at 40° C. The mixture is heated to about 120° C. to 180° C. The reaction zone is under a pressure sufficient to maintain the water in the liquid state. The reaction zone is also under mixing conditions (e.g., turbulent mixing conditions) sufficient to obtain adequate contact between the reactants for a period of time sufficient to obtain a substantially complete reaction to form a lithium simple or complex soap. A product stream is continuously withdrawn from the reaction zone. Then additional lubricating oil is introduced into the product stream to give the grease mixture enough fluidity for circulation. The grease mixture is then continuously introduced into a flash chamber or dehydration zone. The dehydration zone is maintained at a temperature ranging from about 100° C. to 240° C. and at an absolute pressure of 101.35 kPa(a) to 68.35 kPa(a). This ranges from about atmospheric to about 33 kPa(a) (10 inches of mercury) vacuum. The mixture is circulated from the bottom to the top of the dehydration zone through a recycle line and a shear valve having a pressure drop across the valve of from 68.9 kPa(g) to 1034.2 kPa(g) (10 psig to 150 psig). A product stream is continuously withdrawn from the dehydration zone and cooled to provide a finished grease composition. Greases made by this process can also contain additives such as, but not limited to, extreme pressure additives, antioxidants, antirust additives, corrosion inhibitors, dyes, antiwear additives, polymers, solid lubricants, and the like.

As used herein, “chemical driving force” is the difference of the chemical potentials of substances undergoing conversion in their initial and final states. The chemical driving force is the tension between substances in the original and final states associated with the conversion. For example, as lithium 12-hydroxystearate is produced in the neutralization reaction, the difference between the melting temperature of lithium 12-hydroxystearate and the melting temperature of methyl-12-hydroxystearate creates a chemical driving force that initiates the nucleation reaction forming the fibrillary network.

In accordance with this disclosure, the continuous production of grease or other similar organogels is provided that includes feeding continuous streams of metal hydroxide (optionally in a solution) and a fatty acid or methyl ester to a contact zone under specific conditions to enable a neutralization reaction and a nucleation reaction.

The neutralization and nucleation reactions are optionally carried out in the presence of mineral oils (Group I, II, or III), synthetic oils (Group IV, V), polymers, borated esters, defoamants, and/or other additives. The fatty acid or methyl ester stream can optionally contain multiple fatty acids or methyl esters, and these streams can be added/contacted in different areas of the continuous unit to optimize cross-linking or fiber formation of the resultant grease thickener. After neutralization and nucleation, the reaction product optionally undergoes a phase separation process to remove volatile liquids such as water, methanol, or other volatile species. After volatiles are optionally removed from the reaction product, the product is optionally combined with mineral oils (Group I, II, or III), synthetic oils (Group IV, V), polymers, borated esters, defoamants, and/or other additives.

In an embodiment, the present disclosure provides a continuous process for preparing a lithium simple or complex grease, in which the C₃ to C₃₀ hydroxy fatty acid is 12-hydroxy stearic acid; and the C₃ to C₃₀ hydroxy fatty acid ester is the methyl or ethyl ester of 12-hydroxy stearic acid.

In another embodiment, the present disclosure provides a continuous process for preparing a lithium simple or complex grease, in which the temperature of the reaction zone is between about 100° C. to about 240° C. and the pressure is between about 75 psig to about 200 psig; or the temperature of the reaction zone is between about 120° C. to about 220° C.; or the temperature of the reaction zone is between about 140° C. to about 200° C.; or the pressure in the reaction zone is between about 100 psig to about 180 psig; or the pressure in the reaction zone is between about 125 psig to about 175 psig.

In yet another embodiment, the present disclosure provides a continuous process for preparing a lithium simple or complex grease, in which the lithium base is selected from lithium oxide, lithium hydroxide, lithium hydroxide monohydrate and lithium carbonate; or preferably the lithium base is lithium hydroxide.

In still another embodiment, the present disclosure provides a continuous process for preparing a lithium simple or complex grease, in which the base oil is a Group I, Group II, Group III, Group IV, Group V base oil, and combinations thereof.

In an embodiment, the present disclosure provides a continuous process for preparing a lithium simple or complex grease employing an aqueous solution of lithium base, in which the aqueous solution of lithium base is between about 12 wt % to about 24 wt % aqueous lithium hydroxide; or the aqueous solution of lithium base is between about 14 wt % to about 22 wt % aqueous lithium hydroxide; or the lithium hydroxide solution is added in the required stoichiometric amount plus 0.1 wt % excess.

In another embodiment, the present disclosure provides a continuous process for preparing a lithium simple or complex grease, in which mixing is maintained in the reaction zone by recirculating the product in the reaction zone; prior to entering the reaction zone, a heat exchanger raises temperature of the mixture between about 140° C. to about 200° C. at a pressure between about 10 psig to about 40 psig; or prior to entering the reaction zone, a heat exchanger raises temperature of the mixture between about 150° C. to about 190° C.

In yet another embodiment, the present disclosure provides a continuous process for preparing a lithium simple or complex grease, in which the flash chamber zone temperature is between about 100° C. to about 240° C. and the pressure is between about 10 psig to about 20 psig; or the flash chamber zone temperature is between about 120° C. to about 220° C. and the pressure is between about 10 psig to about 20 psig.

In still another embodiment, the present disclosure provides a continuous process for preparing a lithium simple or complex grease, in which the recirculation rate in the reaction zone is such that the volume of recycled mixture during the residence time of said mixture within said reaction zone equals at least 5 to 40 times the total average volume of said mixture within said reaction zone; and the recirculation rate in the flash chamber zone is such that the volume of recycled mixture during the residence time of said mixture within said flash chamber zone equals at least 5 to 40 times the total average volume of said mixture within said flash chamber zone.

In an embodiment, the present disclosure provides a continuous process for preparing a lithium simple or complex grease, in which the grease additives comprise one or more of an antioxidant, a tackiness agent, a viscosity modifier, a nonionic surfactant, a corrosion inhibitor, a rust inhibitor, a wear inhibitor, an extreme pressure agent, and a polymeric compound; or the additives are added as separate mixtures comprising: (i) about 25 to about 35 wt % viscosity modifier, about 12 to about 20 wt % borated additive, about 40 to about 60 wt % of a Group II base oil of viscosity of about 100 to about 120 cSt at 40° C.; (ii) about 30 to about 45 wt % tackiness agent, about 20 to about 30 wt % extreme pressure agent, about 40 to about 60 wt % of a Group II base oil of viscosity of between about 100 and 120 cSt at 40° C.; and (iii) about 30 to about 40 wt % rust inhibitor, about 50 to about 75 wt % of a Group II base oil of viscosity of about 100 to about 120 cSt at 40° C.

In another embodiment, the present disclosure provides a continuous process for preparing a lithium simple or complex grease, in which the mixture from the flash chamber zone is cooled by adding an amount of base oil.

In yet another embodiment, the present disclosure provides a continuous process for preparing a lithium simple or complex grease, in which the process further comprises recycling the formulated grease composition formed in the reaction zone through a reaction zone recycle pump and back through the reaction zone; and the process further comprises recycling the formulated grease composition formed in the flash chamber zone through a flash chamber recycle valve and a flash chamber recycle pump and back through the flash chamber zone.

In still another embodiment, the present disclosure provides a continuous process for preparing a lithium simple or complex grease, in which the amount of base oil added to the mixture is at a rate necessary to give the desired consistency to the formulated grease composition.

In an embodiment, the present disclosure provides a continuous process for preparing a lithium simple or complex grease, wherein after exiting the flash chamber zone, the process further comprises that a temperature of the formulated grease composition is low enough for safe handling and packaging and high enough to permit pumping of the product without excessive pressure drop over the length of the pumping line.

In another embodiment, the present disclosure provides a continuous process for preparing a lithium simple or complex grease, in which the process conditions afford a production throughput of greater than about 8000 lb/hour, 9000 lb/hour or 10,000 lb/hour; or preferably the process conditions afford a production throughput of about 12,000 lb/hour.

In yet another embodiment, the present disclosure provides a continuous process for preparing a lithium simple or complex grease, in which the process conditions afford a formulated grease composition comprising: between about 1.0 wt % to about 1.5 wt % of lithium hydroxide monohydrate; between about 5 wt % to about 10 wt % of methyl 12-hydroxystearate; between about 2.5 wt % to about 7.5 wt % of an extreme pressure agent; between about 2.5 wt % to about 7.5 wt % of a tackiness agent; between about 2.5 wt % to about 5.0 wt % of a viscosity modifier; between about 1 wt % to about 3.5 wt % of a borated additive; between about 0.1 wt % to about 1 wt % of a rust inhibitor; and between about 60 wt % to about 90 wt % of base oil.

The metal base employed in the saponification may be a hydroxide or other suitable basic reacting compound of any of the metals ordinarily employed as the metal component of the soap in the preparation of lubricating greases, such as sodium, lithium, potassium, calcium, barium, magnesium, zinc, cobalt, manganese, aluminum, lead, etc. as well as mixtures of two or more metals. It is preferably a metal oxide, hydroxide or carbonate. The greases which are most advantageously prepared by the method of this disclosure are those wherein the soap thickener is an alkali metal or alkaline earth metal soap, or a mixture of two or more soaps of these classes.

Suitable saponifiable materials for use in these grease preparations comprise fatty acids containing from about 3 to about 30 carbon atoms, preferably higher fatty acids containing from about 12 to about 32 carbon atoms per molecule and hydroxy substituted higher fatty acids, their glycerides and other esters and mixtures thereof. The disclosure also contemplates grease preparations carried out in the manner described above wherein such higher fatty acid materials are employed in conjunction with lower fatty acid materials, such as fatty acids containing from 1 to about 6 carbon atoms per molecule, their glycerides and other esters. Such lower fatty acid materials may be employed in amounts giving a mol ratio of lower fatty acid to higher fatty acid from below 1:1 up to about 20:1, respectively. Also, intermediate fatty acid materials may be employed in conjunction with the higher fatty acid materials in varying amounts, ordinarily in amounts giving a mol ratio with the higher fatty acid material below about 1:1, respectively.

The base oils employed in these greases may be any base oil having lubricating characteristics, including one or more of a Group I, Group II, Group III, Group IV, Group V base oil, and combinations thereof. They may be either naphthenic or paraffinic in type, or blends of two or more oils of these different types.

In the production of greases from synthetic oils which are hydrolyzed under the saponification conditions, the saponification is preferably carried out in the absence of any such base oil or of a minor amount of such base oil which is substantially inert under the saponification conditions such as a mineral oil and the synthetic oil added at later stages of the grease making process described herein.

Base oils are the most important component of grease compositions, generally comprising greater than 70% of the grease compositions. Grease compositions comprise a base oil and at least one additive. Grease compositions can be used in automobiles, diesel engines, axles, transmissions, and industrial applications. Grease compositions must meet the specifications for their intended application as defined by the concerned governing organization. Typical additives and their function are described in Modern Lubricating Greases by C. J. Boner, Scientific Publication (G.B.) Ltd. 1976.

A detailed description of base oils useful in the continuous process of this disclosure is set forth hereinbelow.

Additives, which can be blended with the base oil, to provide a lubricant grease composition include those which are intended to improve select properties of the lubricant grease composition. Typical additives include, for example, ionic surfactants, nonionic surfactants, antioxidants, antiwear additives, extreme pressure agents, corrosion inhibitors, rust inhibitors, thickeners (i.e., gelling agents), oiliness agents, metal deactivators, oil bleed inhibitors such as polybutene, shear stability additives, pour point depressants such as polymethyl methacrylate, complex organic nitrogen, and amines, thermal conductive additives, electroconductive agents, elastomeric compatibilizers, viscosity modifiers, friction modifiers, tackiness agents, bactericides and fungicides, polymeric compounds, colorants, and the like. Additives can be added in the form of an additive package, containing various additives.

Other additives useful in the grease compositions of this disclosure include, for example, boundary lubrication additives, metal passivators, antifoam and defoamants, dispersants, detergents, free radical scavengers, chelating agents, ionic liquids, and the like. Additives may be either liquid or solid (incorporated as a slurry), injected separately or in a pre-blend (which could be a single-phase, two-phase or multi-phase mixture). Solid additives may be characterized as any of macro, micron or nanoparticle sized. Additives useful in the grease compositions of this disclosure are more particularly described herein.

Polymers useful in the grease compositions of this disclosure include, for example, those based on monomers of ethylene, propylene, isopropylene, butylene, iso-butylene, butadiene, isoprene, styrene, etc., alone or in combination of 2 or more, formed as block and/or random polymers, star polymers.

Reactive polymers useful in the grease compositions of this disclosure include, for example, those based on monomers of ethylene, propylene, isopropylene, butylene, iso-butylene, butadiene, isoprene, styrene, vinylidine, etc., alone or in combination of 2 or more, formed as block and/or random polymers, star polymers, with any of a carboxylic acid, anhydride, ester, alcohol, amine, amide, imine or imide terminal functionality.

The polymers, reactive polymers, and additives useful in the grease compositions of this disclosure can also include components used as co-thickeners or supplemental thickeners. For example, isostearic acid blends, fatty amides and long chain polymers can all be incorporated during the soap-formation reaction, where they will be bound intimately into the structure of the soap as it forms and the final dehydrated thickener matrix.

Complexing agents useful in the grease compositions of this disclosure include, for example, dibasic acids with alkyl groups having 1 to 14 carbon atoms, boric acid, methyl salicylate, borated additives (e.g. esters from alkyl alcohols, borated dispersants), and combinations of 2 or more.

Other illustrative additives include, for example, flame retardants such as calcium oxide, tackiness agents such as polyisobutylene (PIB), and the like.

A detailed description of additives useful in the continuous process of this disclosure is set forth hereinbelow.

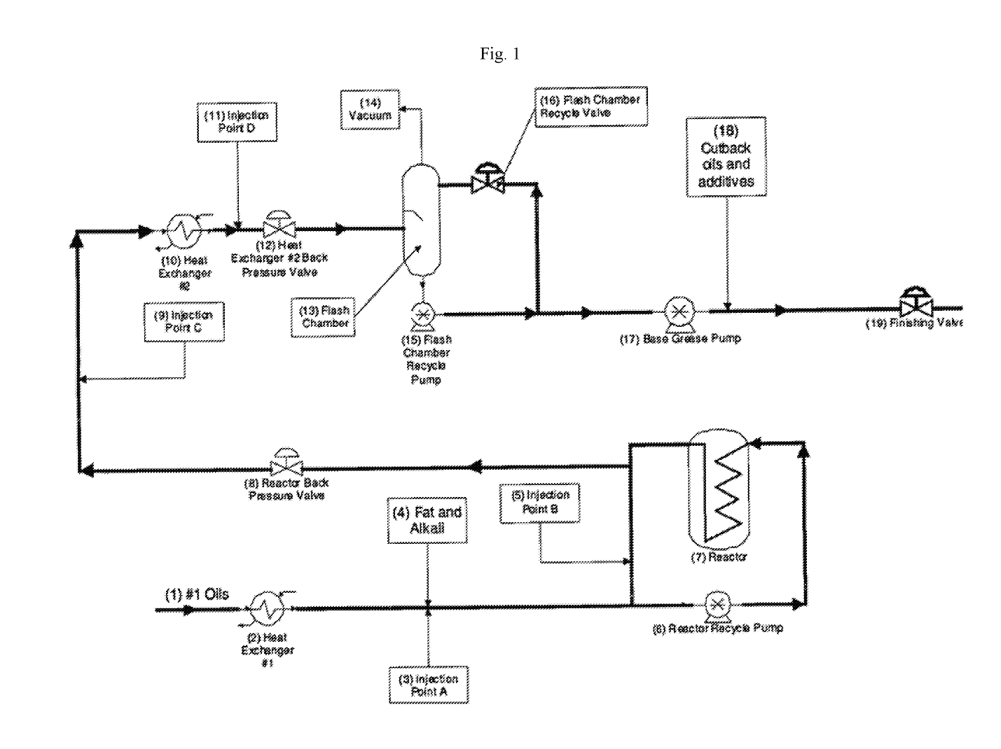

In accordance with this disclosure, a fatty acid and/or methyl ester is heated at a temperature sufficient to react with a metal hydroxide to form a grease thickener. FIG. 1 shows a general flow diagram of an illustrative ILGU of this disclosure.

Referring to FIG. 1, preferred temperature operating conditions are:

### Heat Exchanger #1 (2) 100-240° C.

### Reactor (7) 100-240° C.

### Heat Exchanger #2 (10) 100-240° C.

### Flash Chamber (13) 100-240° C.

Also, referring to FIG. 1, preferred pressure operating conditions are:

Reactor Back Pressure Valve (8) 10-300 psig
Heat Exchanger #2 Back Pressure Valve (12) 10-300 psig
Flash Chamber Recycle Valve (16) 10-300 psig
Finishing or Shear Valve (19) 10-300 psig

Further, referring to FIG. 1, recycle pumps are sized to achieve recycle rates of:

Reactor Recycle Pump (6) 0.5-20 pounds per minute
Flash Chamber Recycle Pump (15) 0.5-20 pounds per minute

The mineral oils (e.g., Group I, II or III base oils) or synthetic oils (e.g., Group IV and V base oils) can be added in stream (1) and stream (18). The mineral oils and synthetic oils can also be added as diluents in stream (3), stream (4), stream (5), stream (9), and stream (11). Base oils useful in the grease compositions of this disclosure are more particularly described herein.

The process reactants including the fatty acid and/or methyl ester and the metal hydroxide can be added in stream (4). The process reactants can also be added in stream (5), stream (9), or stream (11).

The grease additives including borated esters, polymers, and other additives can be added in stream (18). The grease additives can also be added in stream (3), stream (5), stream (9), and stream (11). Borated additives can be useful for improving the high-temperature properties of lithium based grease compositions. For instance, examples of borated additives may be found in U.S. Pat. Nos. 4,780,227; 4,743,386; 4,781,850; 4,828,732; 4,828,734; 5,242,045; 5,242,610; and 5,252,237.

Other grease additives including reactive polymers, defoamants, and other additives can be added in stream (3). The other grease additives can also be added in stream (5), stream (9), and stream (11).

The complexing agents can be added in stream (9) or stream (11). The complexing agent can also be added in stream (3), stream (5), and stream (18).

The mineral oils (e.g., Group I, II or III base oils) or synthetic oils (e.g., Group IV and V base oils) can be added in stream (1) and stream (18). The mineral oils and synthetic oils can also be added as diluents in stream (3), stream (4), stream (5), stream (9), and stream (11). Base oils useful in the grease compositions of this disclosure are more particularly described herein. When added in stream (1), the one or more base oils can pass through heat exchanger #1 (2), which is operated at about 100° C. to about 240° C., prior to passing into the reaction zone (7).

The process reactants including the fatty acid and/or methyl ester and the metal hydroxide can be added in stream (4). The process reactants can also be added in stream (5), stream (9), or stream (11).

The fatty acid and/or methyl ester, or a mixture of fatty acid and/or methyl ester and base oil, which is maintained at a temperature above the melting point of the fatty acid and/or methyl ester by heating, can be added in stream (4). The mixture can be fatty acid and/or methyl ester and base oil comprising at least 10 wt % of the mixture. It is generally preferred to employ a mixture comprising about 20 wt % to 60 wt % of fatty acid and/or methyl ester, although lower amounts down to about 5 wt % and also higher amounts up to 100 wt % of saponifiable material may be employed in some cases. The metal hydroxide can be added as a water solution or oil slurry.

The grease additives including borated esters, polymers, and other additives can be added in stream (18). The grease additives can also be added in stream (3), stream (5), stream (9), and stream (11).

Other grease additives including reactive polymers, defoamants, and other additives can be added in stream (3). The other grease additives can also be added in stream (5), stream (9), and stream (11).

When the saponification is carried out employing a slurry of the metal base in oil, it is generally desirable to introduce a small amount of water or steam into the reaction zone in order to promote the reaction. The reaction mixture in reaction zone (7) is maintained under superatmospheric pressure at least sufficient to maintain the water and methanol present or produced in the reaction in the liquid phase, and at an elevated temperature sufficient to obtain a rapid reaction between the metal base and the saponifiable material. Suitable reaction conditions include broadly pressures in the range from about 10 psig to about 300 psig and temperatures from about 100° C. to about 240° C. The preferred conditions include pressures in the range from about 95 psig to about 170 psig and temperatures in the range from about 160° C. to about 200° C.

The reactant stream is passed through reaction zone (7) at a velocity which is preferably sufficient to maintain flow within the zone. In the process comprising a preferred embodiment of this disclosure, the saponification mixture is recycled continuously through reaction zone (7) by way of reaction zone recycle pump (6), which is operated at about 0.5 to about 20 pounds per minute, as a means of obtaining a sufficiently high rate of flow of the reactant stream through the saponification zone. In this manner, a high rate of flow through reaction zone 1 is maintained which is not dependent upon the feed rate, and controlled flow through the reaction zone can therefore be maintained even with a saponification mixture requiring a relatively long residence time for substantially complete reaction. The recycle rate employed is ordinarily in the ratio from about 10:1 to about 100:1 with the rate of throughput, although somewhat lower or higher recycling ratio may be employed in some cases, such as recycle ratio as low as about 1:1 and as high as about 200:1.

Saponification products obtained under the above conditions are especially suitable for use in the subsequent grease making steps of the process because of the readiness with which they accept additional base oil and the shorter soap conditioning periods which they require as compared with grease mixtures obtained under and other saponification conditions. The different physical conditions of these products are shown by the fact that they form grease-like products immediately upon cooling when the saponification mixture contains base oil, differently from saponification products obtained under different conditions.

A product stream from reaction zone (7) passes through a reaction zone back pressure valve (8), which is operated between about 10 psig and about 300 psig, a heat exchanger #2 (10), which is operated at about 100° C. to about 240° C., and a heat exchanger back pressure valve (12), which is operated between about 10 psig and about 300 psig, to flash chamber zone (13). The flash chamber zone (13) may be jacketed or otherwise provided with indirect heating or cooling means. The grease mixture in flash chamber zone (13) is maintained at an elevated temperature between about 100° C. to about 240° C., but below the melting point of the soap present in the grease mixture, and at a pressure substantially lower than that in reaction zone (7), suitably under a partial vacuum of from about 5 to about 25 inches of mercury. In the preparation of lithium 12-hydroxystearate thickened greases, the grease mixture in flash chamber zone (13) is preferably maintained at a temperature in the range from about 140° C. to about 200° C.

During its residence in flash chamber zone (13), the grease mixture is recycled continuously through a flash chamber recycle pump (15), which is operated at about 0.5 to about 20 pounds per minute, and flash chamber recycle valve (16), which is operated between about 10 psig and about 300 psig. The recycling is preferably carried out at a rapid rate, such that the volume of recycled grease mixture is equal to the total average volume of grease mixture within flash chamber zone (13) (1 batch turnover) within one minute, and sufficient to provide at least about 5 batch turnovers, and most advantageously at least 10 batch turnovers, during the average residence time of the grease mixture within the flash chamber zone (13). The grease residence time in flash chamber zone (13) may be only sufficient to obtain substantially complete dehydration of the grease mixture, the soap conditioning step in this case taking place simultaneously with the final dehydration stages. It is ordinarily prolonged somewhat so as to provide an additional soap conditioning period, preferably for at least about 5 minutes, particularly when the dehydration is accomplished substantially entirely in the initial flashing operation. In carrying out the process under the preferred conditions, the residence time of the grease mixture in flash chamber zone (13) may be from a few minutes up to about 1 hour, depending chiefly upon the character of the soap in the grease mixture, and to a less extent upon other factors such as temperature, soap concentration of the grease mixture and character of the base oil. In the preparation of lithium soap thickened greases, a suitable residence time of the grease mixture in flash chamber zone (13) will usually be from about 5 to about 20 minutes, although somewhat shorter or longer periods may be employed in some cases.

As described herein, additional base oil may be added to the grease mixture at various steps in the process in order to obtain the desired soap concentration or to assist in heating or cooling the grease mixture. Such oil addition may also be employed as a means of heating the grease mixture in order to increase the water removal when the grease mixture is flashed into flash chamber zone (13).

Additionally or alternatively to the base oil addition in the above manner, base oil may be added to the grease mixture during the soap conditioning step. With special advantage in some cases, it may pass into the recycle stream of grease mixture in flash chamber zone (13) as the means of aiding in the recycling when a heavy grease mixture is being circulated, and also as a means of increasing the rate of dehydration by increasing the temperature of the recycle stream in some cases. Additional oil is added in the above manner as required to provide a grease mixture in flash chamber zone (13) containing at least about 25 wt % of base oil, and preferably at least about 40 wt % of base oil.

In addition to the function of the base oil addition to the grease mixture in flash chamber zone (13) as a means of obtaining the desired soap concentration, the oil addition may be employed as a means of either heating or cooling the grease mixture to a temperature within the desired soap conditioning temperature range. When the saponification is carried out at a temperature above the melting point of the soap, the oil added to the grease mixture in flash chamber zone (13) is preferably at a lower temperature than the grease mixture leaving the saponification zone. When the saponification is carried out at a temperature below the desired temperature range for the soap conditioning treatment, the base oil added as described above is preferably preheated. The temperature of the base oil and the amount added may be adjusted so as to give the desired soap concentration in the grease mixture in flash chamber zone (13) and also to provide a temperature within the desired soap conditioning temperature range.

Indirect heating or cooling of the grease mixture in flash chamber zone (13) may be employed either in addition or alternatively to the heating or cooling obtained by oil addition as described above. The indirect heating or cooling may be obtained conveniently by employing a jacketed vessel for flash chamber zone (13) and passing a heat exchange fluid through the vessel jacket.

A stream of substantially dehydrated grease mixture is continuously withdrawn from the recycle stream in flash chamber zone (13) through a base grease pump (17) and finishing or shear valve (19), which is operated between about 10 psig and about 300 psig. Additional base oil may be added to the grease mixture in stream (18). It is ordinarily preferable to add this oil at a temperature substantially lower than that of the grease mixture, very suitably in some cases at ambient temperature. However, in many cases it is advantageous to preheat the oil by passing it through a heat exchanger, particularly where a high rate of oil addition is employed or where it is desirable to employ a slower cooling rate.

The additional oil added to the grease mixture may amount to as much as about 90 wt % of the total oil in the finished grease. It is ordinarily preferable to carry out the grease preparation with about 20-80 wt % of the total oil contained in the grease added in this manner.

A finishing or shear valve (19), such as a gate valve operated with a substantial pressure drop, is used in the continuous process. The stream of grease mixture containing oil added passes through a shearing valve (19). It is ordinarily advantageous to recycle the grease mixture through the shear valve operated with a pressure drop in about the range 20-200 psig, employing a recycle ratio from about 1:1 to about 100:1 and preferably from about 5:1 to about 50:1. Shearing in this manner is preferably carried out upon the grease mixture at a temperature below about 150° C., and most advantageously in most cases at a temperature within the range from about 65° C. to about 125° C.

Any additives employed in the grease are preferably introduced into the grease mixture during the cooling, ordinarily where the grease mixture is below about 125° C. As shown in FIG. 1, the additives preferably may be added in stream (18). The grease additives can also be added in stream (3), stream (5), stream (9), and stream (11).

When simple lithium soaps are manufactured, the soap usually makes up about 5-7 wt % of the final grease for an National Lubricating Grease Institute (NLGI) 2 grade grease. When lithium complex soaps are manufactured, the soap usually makes up about 8-14 wt % of the final grease. Because of this, the complex greases are much heavier as they are manufactured and the concentration of water (both from initial charge and byproducts) and methanol are higher than in simple lithium greases.

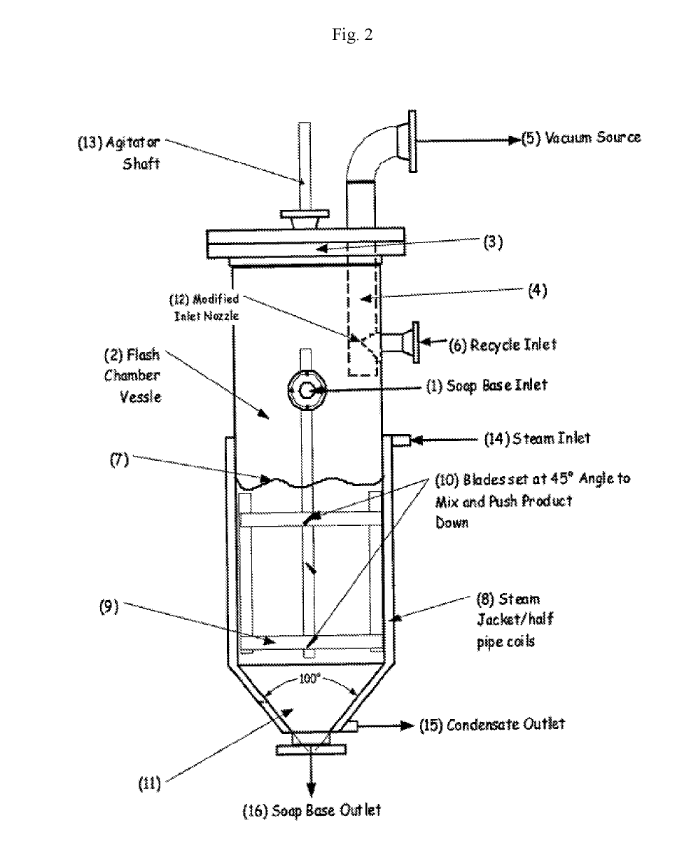

An illustrative flash chamber that efficiently removes the volatile components of lithium simple and complex greases is shown in FIG. 2. Such a flash chamber is described in U.S. Pat. No. 7,829,512, which is incorporated herein by reference in its entirety.

Referring to FIG. 2, the preferred flash chamber has a cylindrical vessel (2) with a length to inner diameter ratio of greater than or equal to 3.2:1, alternatively 3.2:1 to 2.1:1, and alternatively 2.625:1.

The cylindrical vessel (2) has a covered top end (3) and a downwardly sloped conical shaped bottom end (11). The covered top end (3) preferably can be opened for cleaning. The bottom end (11) has an included angle greater than or equal to 90°, alternatively 90° to 120°, alternatively 95° to 105°, and alternatively 100°. This wide angle helps prevent bridging of the soap base as it slumps to the bottom of the chamber. The angle is measured inside face to opposing inside face of the cone. The largest diameter of the cone is proximate to the bottom of the flash chamber. The smallest diameter of the cone terminates in a soap base outlet (16) for removal of an at least partially devolatilized reaction product.

The flash chamber has a soap base inlet (1) for introducing an at least partially saponified reaction product into the vessel (2). A recycle inlet (6) introduces at least partially saponified reaction product through an inlet nozzle (12) into the vessel (2). A vacuum source (5) partially evacuates the vessel (2) through a conduit (4).

The usual upper level of the soap base (7) normally does not foam above the soap base inlet (1) except for potential surges that should always be below the vacuum source (5). This inlet nozzle (12) forces at least partially devolatilized reaction product downwardly into the vessel towards the bottom of the flash chamber. The structure of the inlet nozzle (12), and recycle inlet (6) preferably is downward directed to help knock down existing foam in the flash chamber. The soap base inlet (1), recycle inlet (6), inlet nozzle (12), conduit (4), and vacuum source (5) are in the side or top of the cylindrical vessel above the normal level of the grease (7).

An agitator (13) is positioned within the vessel above the conical shaped bottom end (11). The agitator (13) is provided with a plurality of blades (10) oriented at 15° to 75° (from vertical), alternatively 30° to 55°, alternatively 42° to 48°, and alternatively 45°, forces saponified reaction product downwardly under conditions of use. Preferably the agitator includes a paddle (9) which optionally may touch or hold the blades (10).

A heat transfer mechanism (8) can be added around at least part of the exterior of the cylindrical vessel. Preferably this is a steam heat jacket to improve heating capability.

When used in an ILGU process, the flash chamber is preferably operated at about 100° C. to about 240° C., preferably about 175° C. to about 200° C., alternatively 175° C. to 196° C., and alternatively 180° C. to 190° C. The flash chamber is operated under a sufficient vacuum to at least partially remove the volatiles from the saponified reaction product. This is an absolute pressure of about 88 kPa(a) to about 101 kPa(a) and alternatively an absolute pressure of about 90 kPa(a) to about 100 kPa(a).

A flow meter can be placed in the reactor loop and flash chamber loop to check for changes in flow rate in the loop due to clogging or pump failure.

The current disclosure describes a method of producing complex greases via continuous in-line process that exhibit excellent structural stability, which is at least equivalent to conventional batch processes.

Lithium simple greases made by the process of this disclosure preferably have a penetration split (60X-UW) of within −20 mm/10 to +10 mm/10, a roll stability (D1831) of less than 30 mm/10 (preferably less than 20 mm/10), and a dropping point (D2265) of greater than 240° C. Greases made by the process of this disclosure with a base oil with a viscosity of 90 to 110 cSt @ 40° C. and a fatty matter wt (FM %) within 13% to 13.5%, preferably also have a 60X worked penetration (D1403) of less than 280 mm/10.

Lithium complex greases made by the process of this disclosure preferably have a penetration split (60X-UW) of within −20 mm/10 to +10 mm/10, a roll stability (D1831) of less than 30 mm/10 (preferably less than 20 mm/10), and a dropping point (D2265) of greater than 240° C. Greases made by the improved process of this disclosure with a base oil with a viscosity of 90 to 110 cSt @ 40° C. and a fatty matter wt % (FM %) within 13% to 13.5%, preferably also have a 60X worked penetration (D1403) of less than 280 mm/10.

In the ASTM D2265 dropping point, simple lithium greases are generally less than 180° C. and lithium complexes are generally greater than 240° C.

### Base Oils

A wide range of lubricating base oils is known in the art. Base oils that are useful in the present disclosure are natural oils, mineral oils and synthetic oils, and unconventional oils (or mixtures thereof) can be used unrefined, refined, or rerefined (the latter is also known as reclaimed or reprocessed oil). Unrefined oils are those obtained directly from a natural or synthetic source and used without added purification. These include shale oil obtained directly from retorting operations, petroleum oil obtained directly from primary distillation, and ester oil obtained directly from an esterification process. Refined oils are similar to the oils discussed for unrefined oils except refined oils are subjected to one or more purification steps to improve at least one base oil property. One skilled in the art is familiar with many purification processes. These processes include solvent extraction, secondary distillation, acid extraction, base extraction, filtration, and percolation. Rerefined oils are obtained by processes analogous to refined oils but using an oil that has been previously used as a feed stock.

Groups I, II, III, IV and V are broad base oil stock categories developed and defined by the American Petroleum Institute (API Publication 1509; www.API.org) to create guidelines for lubricant base oils. Group I base stocks have a viscosity index of between about 80 to 120 and contain greater than about 0.03% sulfur and/or less than about 90% saturates. Group II base stocks have a viscosity index of between about 80 to 120, and contain less than or equal to about 0.03% sulfur and greater than or equal to about 90% saturates. Group III stocks have a viscosity index greater than about 120 and contain less than or equal to about 0.03% sulfur and greater than about 90% saturates. Group IV includes polyalphaolefins (PAO). Group V base stock includes base stocks not included in Groups I-IV. The table below summarizes properties of each of these five groups.

| | Base Oil Properties | | |
|---|---|---|---|
| | Saturates | Sulfur | Viscosity Index |
| Group I | <90 and/or | >0.03% and | ≥80 and <120 |
| Group II | ≥90 and | ≤0.03% and | ≥80 and <120 |
| Group III | ≥90 and | ≤0.03% and | ≥120 |

| | |
|---|---|
| Group IV | polyalphaolefins (PAO) |
| Group V | All other base oil stocks not included in Groups I, II, III or IV |

Natural oils include animal oils, vegetable oils (castor oil and lard oil, for example), and mineral oils. Animal and vegetable oils possessing favorable thermal oxidative stability can be used. Of the natural oils, mineral oils are preferred. Mineral oils vary widely as to their crude source, for example, as to whether they are paraffinic, naphthenic, or mixed paraffinic-naphthenic. Oils derived from coal or shale are also useful. Natural oils vary also as to the method used for their production and purification, for example, their distillation range and whether they are straight run or cracked, hydrorefined, or solvent extracted.

Group II and/or Group III hydroprocessed or hydrocracked base stocks are also well known base stock oils.

Synthetic oils include hydrocarbon oil. Hydrocarbon oils include oils such as polymerized and interpolymerized olefins (polybutylenes, polypropylenes, propylene isobutylene copolymers, ethylene-olefin copolymers, and ethylene-alphaolefin copolymers, for example). Polyalphaolefin (PAO) oil base stocks are commonly used synthetic hydrocarbon oil. By way of example, PAOs derived from C₈, C₁₀, C₁₂, C₁₄ olefins or mixtures thereof may be utilized. See U.S. Pat. Nos. 4,956,122; 4,827,064; and 4,827,073.

The number average molecular weights of the PAOs, which are known materials and generally available on a major commercial scale from suppliers such as ExxonMobil Chemical Company, Chevron Phillips Chemical Company, BP, and others, typically vary from about 250 to about 3,000, although PAO's may be made in viscosities up to about 150 cSt (100° C.). The PAOs are typically comprised of relatively low molecular weight hydrogenated polymers or oligomers of alphaolefins which include, but are not limited to, C₂ to about C₃₂ alphaolefins with the C₈ to about C₁₆ alphaolefins, such as 1-octene, 1-decene, 1-dodecene and the like, being preferred. The preferred polyalphaolefins are poly-1-octene, poly-1-decene and poly-1-dodecene and mixtures thereof and mixed olefin-derived polyolefins. However, the dimers of higher olefins in the range of C₁₂ to C₁₈ may be used to provide low viscosity base stocks of acceptably low volatility. Depending on the viscosity grade and the starting oligomer, the PAOs may be predominantly dimers, trimers and tetramers of the starting olefins, with minor amounts of the lower and/or higher oligomers, having a viscosity range of 1.5 cSt to 12 cSt. PAO fluids of particular use may include 3 cSt, 3.4 cSt, and/or 3.6 cSt and combinations thereof. Mixtures of PAO fluids having a viscosity range of 1.5 cSt to approximately 150 cSt or more may be used if desired. Unless indicated otherwise, all viscosities cited herein are measured at 100° C.

The PAO fluids may be conveniently made by the polymerization of an alphaolefin in the presence of a polymerization catalyst such as the Friedel-Crafts catalysts including, for example, aluminum trichloride, boron trifluoride or complexes of boron trifluoride with water, alcohols such as ethanol, propanol or butanol, carboxylic acids or esters such as ethyl acetate or ethyl propionate. For example the methods disclosed by U.S. Pat. No. 4,149,178 or 3,382,291 may be conveniently used herein. Other descriptions of PAO synthesis are found in the following U.S. Pat. Nos. 3,742,082; 3,769,363; 3,876,720; 4,239,930; 4,367,352; 4,413,156; 4,434,408; 4,910,355; 4,956,122; and 5,068,487. The dimers of the C₁₄ to C₁₈ olefins are described in U.S. Pat. No. 4,218,330.

Other useful lubricant oil base stocks include wax isomerate base stocks and base oils, comprising hydroisomerized waxy stocks (e.g. waxy stocks such as gas oils, slack waxes, fuels hydrocracker bottoms, etc.), hydroisomerized Fischer-Tropsch waxes, Gas-to-Liquids (GTL) base stocks and base oils, and other wax isomerate hydroisomerized base stocks and base oils, or mixtures thereof. Fischer-Tropsch waxes, the high boiling point residues of Fischer-Tropsch synthesis, are highly paraffinic hydrocarbons with very low sulfur content. The hydroprocessing used for the production of such base stocks may use an amorphous hydrocracking/hydroisomerization catalyst, such as one of the specialized lube hydrocracking (LHDC) catalysts or a crystalline hydrocracking/hydroisomerization catalyst, preferably a zeolitic catalyst. For example, one useful catalyst is ZSM-48 as described in U.S. Pat. No. 5,075,269, the disclosure of which is incorporated herein by reference in its entirety. Processes for making hydrocracked/hydroisomerized distillates and hydrocracked/hydroisomerized waxes are described, for example, in U.S. Pat. Nos. 2,817,693; 4,975,177; 4,921,594 and 4,897,178 as well as in British Patent Nos. 1,429,494; 1,350,257; 1,440,230 and 1,390,359. Each of the aforementioned patents is incorporated herein in their entirety. Particularly favorable processes are described in European Patent Application Nos. 464546 and 464547, also incorporated herein by reference. Processes using Fischer-Tropsch wax feeds are described in U.S. Pat. Nos. 4,594,172 and 4,943,672, the disclosures of which are incorporated herein by reference in their entirety.

Gas-to-Liquids (GTL) base oils, Fischer-Tropsch wax derived base oils, and other wax-derived hydroisomerized (wax isomerate) base oils be advantageously used in the instant disclosure, and may have useful kinematic viscosities at 100° C. of about 2 cSt to about 50 cSt, preferably about 2 cSt to about 30 cSt, more preferably about 3 cSt to about 25 cSt, as exemplified by GTL 4 with kinematic viscosity of about 4.0 cSt at 100° C. and a viscosity index of about 141. These Gas-to-Liquids (GTL) base oils, Fischer-Tropsch wax derived base oils, and other wax-derived hydroisomerized base oils may have useful pour points of about −20° C. or lower, and under some conditions may have advantageous pour points of about −25° C. or lower, with useful pour points of about −30° C. to about −40° C. or lower. Useful compositions of Gas-to-Liquids (GTL) base oils, Fischer-Tropsch wax derived base oils, and wax-derived hydroisomerized base oils are recited in U.S. Pat. Nos. 6,080,301; 6,090,989, and 6,165,949 for example, and are incorporated herein in their entirety by reference.

The hydrocarbyl aromatics can be used as a base oil or base oil component and can be any hydrocarbyl molecule that contains at least about 5% of its weight derived from an aromatic moiety such as a benzenoid moiety or naphthenoid moiety, or their derivatives. These hydrocarbyl aromatics include alkyl benzenes, alkyl naphthalenes, alkyl biphenyls, alkyl diphenyl oxides, alkyl naphthols, alkyl diphenyl sulfides, alkylated bis-phenol A, alkylated thiodiphenol, and the like. The aromatic can be mono-alkylated, dialkylated, polyalkylated, and the like. The aromatic can be mono- or poly-functionalized. The hydrocarbyl groups can also be comprised of mixtures of alkyl groups, alkenyl groups, alkynyl, cycloalkyl groups, cycloalkenyl groups and other related hydrocarbyl groups. The hydrocarbyl groups can range from about C₆ up to about C₆₀ with a range of about C₈ to about C₂₀ often being preferred. A mixture of hydrocarbyl groups is often preferred, and up to about three such substituents may be present. The hydrocarbyl group can optionally contain sulfur, oxygen, and/or nitrogen containing substituents. The aromatic group can also be derived from natural (petroleum) sources, provided at least about 5% of the molecule is comprised of an above-type aromatic moiety. Viscosities at 100° C. of approximately 2 cSt to about 50 cSt are preferred, with viscosities of approximately 3 cSt to about 20 cSt often being more preferred for the hydrocarbyl aromatic component. In one embodiment, an alkyl naphthalene where the alkyl group is primarily comprised of 1-hexadecene is used. Other alkylates of aromatics can be advantageously used. Naphthalene or methyl naphthalene, for example, can be alkylated with olefins such as octene, decene, dodecene, tetradecene or higher, mixtures of similar olefins, and the like. Alkylated naphthalene and analogues may also comprise compositions with isomeric distribution of alkylating groups on the alpha and beta carbon positions of the ring structure. Distribution of groups on the alpha and beta positions of a naphthalene ring may range from 100:1 to 1:100, more often 50:1 to 1:50 Useful concentrations of hydrocarbyl aromatic in a lubricant oil composition can be about 2% to about 25%, preferably about 4% to about 20%, and more preferably about 4% to about 15%, depending on the application.

Alkylated aromatics such as the hydrocarbyl aromatics of the present disclosure may be produced by well-known Friedel-Crafts alkylation of aromatic compounds. See Friedel-Crafts and Related Reactions, Olah, G. A. (ed.), Inter-science Publishers, New York, 1963. For example, an aromatic compound, such as benzene or naphthalene, is alkylated by an olefin, alkyl halide or alcohol in the presence of a Friedel-Crafts catalyst. See Friedel-Crafts and Related Reactions, Vol. 2, part 1, chapters 14, 17, and 18, See Olah, G. A. (ed.), Inter-science Publishers, New York, 1964. Many homogeneous or heterogeneous, solid catalysts are known to one skilled in the art. The choice of catalyst depends on the reactivity of the starting materials and product quality requirements. For example, strong acids such as AlCl₃, BF₃, or HF may be used. In some cases, milder catalysts such as FeCl₃ or SnCl₄ are preferred. Newer alkylation technology uses zeolites or solid super acids.

Esters comprise a useful base stock. Additive solvency and seal compatibility characteristics may be secured by the use of esters such as the esters of dibasic acids with monoalkanols and the polyol esters of monocarboxylic acids. Esters of the former type include, for example, the esters of dicarboxylic acids such as phthalic acid, succinic acid, alkyl succinic acid, alkenyl succinic acid, maleic acid, azelaic acid, suberic acid, sebacic acid, fumaric acid, adipic acid, linoleic acid dimer, malonic acid, alkyl malonic acid, alkenyl malonic acid, etc., with a variety of alcohols such as butyl alcohol, hexyl alcohol, dodecyl alcohol, 2-ethylhexyl alcohol, etc. Specific examples of these types of esters include dibutyl adipate, di(2-ethylhexyl) sebacate, di-n-hexyl fumarate, dioctyl sebacate, diisooctyl azelate, diisodecyl azelate, dioctyl phthalate, didecyl phthalate, dieicosyl sebacate, etc.

Particularly useful synthetic esters are those which are obtained by reacting one or more polyhydric alcohols, preferably the hindered polyols (such as the neopentyl polyols, e.g., neopentyl glycol, trimethylol ethane, 2-methyl-2-propyl-1,3-propanediol, trimethylol propane, pentaerythritol and dipentaerythritol) with alkanoic acids containing at least about 4 carbon atoms, preferably C₅ to C₃₀ acids such as saturated straight chain fatty acids including caprylic acid, capric acid, lauric acid, myristic acid, palmitic acid, stearic acid, arachic acid, and behenic acid, or the corresponding branched chain fatty acids or unsaturated fatty acids such as oleic acid, or mixtures of any of these materials.

Suitable synthetic ester components include the esters of trimethylol propane, trimethylol butane, trimethylol ethane, pentaerythritol and/or dipentaerythritol with one or more monocarboxylic acids containing from about 5 to about 10 carbon atoms. These esters are widely available commercially, for example, the Mobil P-41 and P-51 esters of ExxonMobil Chemical Company.

Also useful are esters derived from renewable material such as coconut, palm, rapeseed, soy, sunflower and the like. These esters may be monoesters, di-esters, polyol esters, complex esters, or mixtures thereof. These esters are widely available commercially, for example, the Mobil P-51 ester of ExxonMobil Chemical Company.

Grease compositions containing renewable esters are included in this disclosure. For such formulations, the renewable content of the ester is typically greater than about 70 weight percent, preferably more than about 80 weight percent and most preferably more than about 90 weight percent.

Other useful fluids of lubricating viscosity include non-conventional or unconventional base stocks that have been processed, preferably catalytically, or synthesized to provide high performance lubrication characteristics.

Non-conventional or unconventional base stocks/base oils include one or more of a mixture of base stock(s) derived from one or more Gas-to-Liquids (GTL) materials, as well as isomerate/isodewaxate base stock(s) derived from natural wax or waxy feeds, mineral and or non-mineral oil waxy feed stocks such as slack waxes, natural waxes, and waxy stocks such as gas oils, waxy fuels hydrocracker bottoms, waxy raffinate, hydrocrackate, thermal crackates, or other mineral, mineral oil, or even non-petroleum oil derived waxy materials such as waxy materials received from coal liquefaction or shale oil, and mixtures of such base stocks.

GTL materials are materials that are derived via one or more synthesis, combination, transformation, rearrangement, and/or degradation/deconstructive processes from gaseous carbon-containing compounds, hydrogen-containing compounds and/or elements as feed stocks such as hydrogen, carbon dioxide, carbon monoxide, water, methane, ethane, ethylene, acetylene, propane, propylene, propyne, butane, butylenes, and butynes. GTL base stocks and/or base oils are GTL materials of lubricating viscosity that are generally derived from hydrocarbons; for example, waxy synthesized hydrocarbons, that are themselves derived from simpler gaseous carbon-containing compounds, hydrogen-containing compounds and/or elements as feed stocks. GTL base stock(s) and/or base oil(s) include oils boiling in the lube oil boiling range (1) separated/fractionated from synthesized GTL materials such as, for example, by distillation and subsequently subjected to a final wax processing step which involves either or both of a catalytic dewaxing process, or a solvent dewaxing process, to produce lube oils of reduced/low pour point; (2) synthesized wax isomerates, comprising, for example, hydrodewaxed or hydroisomerized cat and/or solvent dewaxed synthesized wax or waxy hydrocarbons; (3) hydrodewaxed or hydroisomerized cat and/or solvent dewaxed Fischer-Tropsch (F-T) material (i.e., hydrocarbons, waxy hydrocarbons, waxes and possible analogous oxygenates); preferably hydrodewaxed or hydroisomerized/followed by cat and/or solvent dewaxing dewaxed F-T waxy hydrocarbons, or hydrodewaxed or hydroisomerized/followed by cat (or solvent) dewaxing dewaxed, F-T waxes, or mixtures thereof.

GTL base stock(s) and/or base oil(s) derived from GTL materials, especially, hydrodewaxed or hydroisomerized/followed by cat and/or solvent dewaxed wax or waxy feed, preferably F-T material derived base stock(s) and/or base oil(s), are characterized typically as having kinematic viscosities at 100° C. of from about 2 mm²/s to about 50 mm²/s (ASTM D445). They are further characterized typically as having pour points of −5° C. to about −40° C. or lower (ASTM D97). They are also characterized typically as having viscosity indices of about 80 to about 140 or greater (ASTM D2270).

In addition, the GTL base stock(s) and/or base oil(s) are typically highly paraffinic (>90% saturates), and may contain mixtures of monocycloparaffins and multicycloparaffins in combination with non-cyclic isoparaffins. The ratio of the naphthenic (i.e., cycloparaffin) content in such combinations varies with the catalyst and temperature used. Further, GTL base stock(s) and/or base oil(s) typically have very low sulfur and nitrogen content, generally containing less than about 10 ppm, and more typically less than about 5 ppm of each of these elements. The sulfur and nitrogen content of GTL base stock(s) and/or base oil(s) obtained from F-T material, especially F-T wax, is essentially nil. In addition, the absence of phosphorus and aromatics make this materially especially suitable for the formulation of low SAP products.

The term GTL base stock and/or base oil and/or wax isomerate base stock and/or base oil is to be understood as embracing individual fractions of such materials of wide viscosity range as recovered in the production process, mixtures of two or more of such fractions, as well as mixtures of one or two or more low viscosity fractions with one, two or more higher viscosity fractions to produce a blend wherein the blend exhibits a target kinematic viscosity.

The GTL material, from which the GTL base stock(s) and/or base oil(s) is/are derived is preferably an F-T material (i.e., hydrocarbons, waxy hydrocarbons, wax).

Base oils for use in the grease compositions of the present disclosure are any of the variety of oils corresponding to API Group I, Group II, Group III, Group IV, and Group V oils and mixtures thereof, preferably API Group II, Group III, Group IV, and Group V oils and mixtures thereof, more preferably the Group III to Group V base oils due to their exceptional volatility, stability, viscometric and cleanliness features. Minor quantities of Group I stock, such as the amount used to dilute additives for blending into formulated lube oil products, can be tolerated but should be kept to a minimum, i.e. amounts only associated with their use as diluent/carrier oil for additives used on an “as-received” basis. Even in regard to the Group II stocks, it is preferred that the Group II stock be in the higher quality range associated with that stock, i.e. a Group II stock having a viscosity index in the range 100<VI<120.

The base oil constitutes the major component of the grease composition of the present disclosure and typically is present in an amount ranging from about 6 to about 99 weight percent or from about 6 to about 95 weight percent, preferably from about 50 to about 99 weight percent or from about 70 to about 95 weight percent, and more preferably from about 85 to about 95 weight percent, based on the total weight of the composition. The base oil may be selected from any of the synthetic or natural oils. The base oil conveniently has a kinematic viscosity, according to ASTM standards, of about 2.5 cSt to about 18 cSt (or mm²/s) at 100° C. and preferably of about 2.5 cSt to about 12.5 cSt (or mm²/s) at 100° C., often more preferably from about 2.5 cSt to about 10 cSt. Mixtures of synthetic and natural base oils may be used if desired. Bi-modal, tri-modal, and additional combinations of mixtures of Group I, II, III, IV, and/or V base stocks may be used if desired.

### Grease Additives

The grease compositions of the present disclosure may additionally contain one or more of the commonly used performance additives including but not limited to antiwear additives, dispersants, detergents, viscosity modifiers, antioxidants, corrosion inhibitors, rust inhibitors, metal deactivators, extreme pressure additives, anti-seizure agents, wax modifiers, viscosity modifiers, fluid-loss additives, seal compatibility agents, anti-staining agents, chromophoric agents, defoamants, demulsifiers, densifiers, wetting agents, gelling agents, tackiness agents, colorants, ionic liquids, metal passivators, and others. These additives are commonly delivered with varying amounts of diluent oil, that may range from 5 weight percent to 50 weight percent.

The additives useful in this disclosure do not have to be soluble in the grease compositions. Insoluble additives in oil can be dispersed in the grease compositions of this disclosure.

The types and quantities of performance additives used in combination with the instant disclosure in grease compositions are not limited by the examples shown herein as illustrations.

### Antiwear Additives

A metal alkylthiophosphate and more particularly a metal dialkyl dithio phosphate in which the metal constituent is zinc, or zinc dialkyl dithio phosphate (ZDDP) can be a useful component of the grease compositions of this disclosure. ZDDP can be derived from primary alcohols, secondary alcohols or mixtures thereof. ZDDP compounds generally are of the formula

Zn[SP(S)(OR¹)(OR²)]₂

where R¹ and R² are C₁-C₁₈ alkyl groups, preferably C₂-C₁₂ alkyl groups. These alkyl groups may be straight chain or branched. Alcohols used in the ZDDP can be propanol, 2-propanol, butanol, secondary butanol, pentanols, hexanols such as 4-methyl-2-pentanol, n-hexanol, n-octanol, 2-ethyl hexanol, alkylated phenols, and the like. Mixtures of secondary alcohols or of primary and secondary alcohol can be preferred. Alkyl aryl groups may also be used.

Preferable zinc dithiophosphates which are commercially available include secondary zinc dithiophosphates such as those available from for example, The Lubrizol Corporation under the trade designations “LZ 677A”, “LZ 1095” and “LZ 1371”, from for example Chevron Oronite under the trade designation “OLOA 262” and from for example Afton Chemical under the trade designation “HITEC 7169”.

The ZDDP is typically used in amounts of from about 0.3 weight percent to about 1.5 weight percent, preferably from about 0.4 weight percent to about 1.2 weight percent, more preferably from about 0.5 weight percent to about 1.0 weight percent, and even more preferably from about 0.6 weight percent to about 0.8 weight percent, based on the total weight of the grease composition, although more or less can often be used advantageously. Preferably, the ZDDP is a secondary ZDDP and present in an amount of from about 0.6 to 1.0 weight percent of the total weight of the grease composition.

### Dispersants

During operation, oil-insoluble oxidation byproducts are produced. Dispersants help keep these byproducts in solution, thus diminishing their deposition on metal surfaces. Dispersants used in the grease compositions may be ashless or ash-forming in nature. Preferably, the dispersant is ashless. So called ashless dispersants are organic materials that form substantially no ash upon combustion. For example, non-metal-containing or borated metal-free dispersants are considered ashless. In contrast, metal-containing detergents discussed above form ash upon combustion.

Suitable dispersants typically contain a polar group attached to a relatively high molecular weight hydrocarbon chain. The polar group typically contains at least one element of nitrogen, oxygen, or phosphorus. Typical hydrocarbon chains contain 50 to 400 carbon atoms.

A particularly useful class of dispersants are the (poly)alkenylsuccinic derivatives, typically produced by the reaction of a long chain hydrocarbyl substituted succinic compound, usually a hydrocarbyl substituted succinic anhydride, with a polyhydroxy or polyamino compound. The long chain hydrocarbyl group constituting the oleophilic portion of the molecule which confers solubility in the oil, is normally a polyisobutylene group. Many examples of this type of dispersant are well known commercially and in the literature. Exemplary U.S. patents describing such dispersants are U.S. Pat. Nos. 3,172,892; 3,2145,707; 3,219,666; 3,316,177; 3,341,542; 3,444,170; 3,454,607; 3,541,012; 3,630,904; 3,632,511; 3,787,374 and 4,234,435. Other types of dispersant are described in U.S. Pat. Nos. 3,036,003; 3,200,107; 3,254,025; 3,275,554; 3,438,757; 3,454,555; 3,565,804; 3,413,347; 3,697,574; 3,725,277; 3,725,480; 3,726,882; 4,454,059; 3,329,658; 3,449,250; 3,519,565; 3,666,730; 3,687,849; 3,702,300; 4,100,082; 5,705,458. A further description of dispersants may be found, for example, in European Patent Application No. 471 071, to which reference is made for this purpose.

Hydrocarbyl-substituted succinic acid and hydrocarbyl-substituted succinic anhydride derivatives are useful dispersants. In particular, succinimide, succinate esters, or succinate ester amides prepared by the reaction of a hydrocarbon-substituted succinic acid compound preferably having at least 50 carbon atoms in the hydrocarbon substituent, with at least one equivalent of an alkylene amine are particularly useful.

Succinimides are formed by the condensation reaction between hydrocarbyl substituted succinic anhydrides and amines. Molar ratios can vary depending on the polyamine. For example, the molar ratio of hydrocarbyl substituted succinic anhydride to TEPA can vary from about 1:1 to about 5:1. Representative examples are shown in U.S. Pat. Nos. 3,087,936; 3,172,892; 3,219,666; 3,272,746; 3,322,670; and U.S. Pat. Nos. 3,652,616, 3,948,800; and Canada Patent No. 1,094,044.

Succinate esters are formed by the condensation reaction between hydrocarbyl substituted succinic anhydrides and alcohols or polyols. Molar ratios can vary depending on the alcohol or polyol used. For example, the condensation product of a hydrocarbyl substituted succinic anhydride and pentaerythritol is a useful dispersant.

Succinate ester amides are formed by condensation reaction between hydrocarbyl substituted succinic anhydrides and alkanol amines. For example, suitable alkanol amines include ethoxylated polyalkylpolyamines, propoxylated polyalkylpolyamines and polyalkenylpolyamines such as polyethylene polyamines. One example is propoxylated hexamethylenediamine. Representative examples are shown in U.S. Pat. No. 4,426,305.

The molecular weight of the hydrocarbyl substituted succinic anhydrides used in the preceding paragraphs will typically range between 800 and 2,500 or more. The above products can be post-reacted with various reagents such as sulfur, oxygen, formaldehyde, carboxylic acids such as oleic acid. The above products can also be post reacted with boron compounds such as boric acid, borate esters or highly borated dispersants, to form borated dispersants generally having from about 0.1 to about 5 moles of boron per mole of dispersant reaction product.

Mannich base dispersants are made from the reaction of alkylphenols, formaldehyde, and amines. See U.S. Pat. No. 4,767,551, which is incorporated herein by reference. Process aids and catalysts, such as oleic acid and sulfonic acids, can also be part of the reaction mixture. Molecular weights of the alkylphenols range from 800 to 2,500. Representative examples are shown in U.S. Pat. Nos. 3,697,574; 3,703,536; 3,704,308; 3,751,365; 3,756,953; 3,798,165; and 3,803,039.

Typical high molecular weight aliphatic acid modified Mannich condensation products useful in this disclosure can be prepared from high molecular weight alkyl-substituted hydroxyaromatics or HNR₂ group-containing reactants.

Hydrocarbyl substituted amine ashless dispersant additives are well known to one skilled in the art; see, for example, U.S. Pat. Nos. 3,275,554; 3,438,757; 3,565,804; 3,755,433, 3,822,209, and 5,084,197.

Preferred dispersants include borated and non-borated succinimides, including those derivatives from mono-succinimides, bis-succinimides, and/or mixtures of mono- and bis-succinimides, wherein the hydrocarbyl succinimide is derived from a hydrocarbylene group such as polyisobutylene having a Mn of from about 500 to about 5000, or from about 1000 to about 3000, or about 1000 to about 2000, or a mixture of such hydrocarbylene groups, often with high terminal vinylic groups. Other preferred dispersants include succinic acid-esters and amides, alkylphenol-polyamine-coupled Mannich adducts, their capped derivatives, and other related components.

Polymethacrylate or polyacrylate derivatives are another class of dispersants. These dispersants are typically prepared by reacting a nitrogen containing monomer and a methacrylic or acrylic acid esters containing 5-25 carbon atoms in the ester group. Representative examples are shown in U.S. Pat. Nos. 2,100,993, and 6,323,164. Polymethacrylate and polyacrylate dispersants are normally used as multifunctional viscosity modifiers. The lower molecular weight versions can be used as dispersants or fuel detergents.

Illustrative preferred dispersants useful in this disclosure include those derived from polyalkenyl-substituted mono- or dicarboxylic acid, anhydride or ester, which dispersant has a polyalkenyl moiety with a number average molecular weight of at least 900 and from greater than 1.3 to 1.7, preferably from greater than 1.3 to 1.6, most preferably from greater than 1.3 to 1.5, functional groups (mono- or dicarboxylic acid producing moieties) per polyalkenyl moiety (a medium functionality dispersant). Functionality (F) can be determined according to the following formula:

F=(SAP×Mₙ)/((112,200×A.I.)−(SAP×98))

wherein SAP is the saponification number (i.e., the number of milligrams of KOH consumed in the complete neutralization of the acid groups in one gram of the succinic-containing reaction product, as determined according to ASTM D94); Mₙ is the number average molecular weight of the starting olefin polymer; and A.I. is the percent active ingredient of the succinic-containing reaction product (the remainder being unreacted olefin polymer, succinic anhydride and diluent).

The polyalkenyl moiety of the dispersant may have a number average molecular weight of at least 900, suitably at least 1500, preferably between 1800 and 3000, such as between 2000 and 2800, more preferably from about 2100 to 2500, and most preferably from about 2200 to about 2400. The molecular weight of a dispersant is generally expressed in terms of the molecular weight of the polyalkenyl moiety. This is because the precise molecular weight range of the dispersant depends on numerous parameters including the type of polymer used to derive the dispersant, the number of functional groups, and the type of nucleophilic group employed.

Polymer molecular weight, specifically Mₙ, can be determined by various known techniques. One convenient method is gel permeation chromatography (GPC), which additionally provides molecular weight distribution information (see W. W. Yau, J. J. Kirkland and D. D. Bly, “Modern Size Exclusion Liquid Chromatography”, John Wiley and Sons, New York, 1979). Another useful method for determining molecular weight, particularly for lower molecular weight polymers, is vapor pressure osmometry (e.g., ASTM D3592).

The polyalkenyl moiety in a dispersant preferably has a narrow molecular weight distribution (MWD), also referred to as polydispersity, as determined by the ratio of weight average molecular weight (M_{w}) to number average molecular weight (Mₙ). Polymers having a M_{w}/Mₙ of less than 2.2, preferably less than 2.0, are most desirable. Suitable polymers have a polydispersity of from about 1.5 to 2.1, preferably from about 1.6 to about 1.8.

Suitable polyalkenes employed in the formation of the dispersants include homopolymers, interpolymers or lower molecular weight hydrocarbons. One family of such polymers comprise polymers of ethylene and/or at least one C₃ to C₂ alpha-olefin having the formula H₂C═CHR¹ wherein R¹ is a straight or branched chain alkyl radical comprising 1 to 26 carbon atoms and wherein the polymer contains carbon-to-carbon unsaturation, and a high degree of terminal ethenylidene unsaturation. Preferably, such polymers comprise interpolymers of ethylene and at least one alpha-olefin of the above formula, wherein R¹ is alkyl of from 1 to 18 carbon atoms, and more preferably is alkyl of from 1 to 8 carbon atoms, and more preferably still of from 1 to 2 carbon atoms.

Another useful class of polymers is polymers prepared by cationic polymerization of monomers such as isobutene and styrene. Common polymers from this class include polyisobutenes obtained by polymerization of a C₄ refinery stream having a butene content of 35 to 75% by wt., and an isobutene content of 30 to 60% by wt. A preferred source of monomer for making poly-n-butenes is petroleum feedstreams such as Raffinate II. These feedstocks are disclosed in the art such as in U.S. Pat. No. 4,952,739. A preferred embodiment utilizes polyisobutylene prepared from a pure isobutylene stream or a Raffinate I stream to prepare reactive isobutylene polymers with terminal vinylidene olefins. Polyisobutene polymers that may be employed are generally based on a polymer chain of from 1500 to 3000.

The dispersant(s) are preferably non-polymeric (e.g., mono- or bis-succinimides). Such dispersants can be prepared by conventional processes such as disclosed in U.S. Patent Application Publication No. 2008/0020950, the disclosure of which is incorporated herein by reference.

The dispersant(s) can be borated by conventional means, as generally disclosed in U.S. Pat. Nos. 3,087,936, 3,254,025 and 5,430,105.

Such dispersants may be used in an amount of about 0.01 to 20 weight percent or 0.01 to 10 weight percent, preferably about 0.5 to 8 weight percent, or more preferably 0.5 to 4 weight percent. Or such dispersants may be used in an amount of about 2 to 12 weight percent, preferably about 4 to 10 weight percent, or more preferably 6 to 9 weight percent. On an active ingredient basis, such additives may be used in an amount of about 0.06 to 14 weight percent, preferably about 0.3 to 6 weight percent. The hydrocarbon portion of the dispersant atoms can range from C₆₀ to C₁₀₀₀, or from C₇₀ to C₃₀₀, or from C₇₀ to C₂₀₀. These dispersants may contain both neutral and basic nitrogen, and mixtures of both. Dispersants can be end-capped by borates and/or cyclic carbonates. Nitrogen content in the finished oil can vary from about 200 ppm by weight to about 2000 ppm by weight, preferably from about 200 ppm by weight to about 1200 ppm by weight. Basic nitrogen can vary from about 100 ppm by weight to about 1000 ppm by weight, preferably from about 100 ppm by weight to about 600 ppm by weight.

Dispersants as described herein are beneficially useful with the compositions of this disclosure. Further, in one embodiment, preparation of the compositions of this disclosure using one or more dispersants is achieved by combining ingredients of this disclosure, plus optional base stocks and grease additives, in a mixture at a temperature above the melting point of such ingredients, particularly that of the one or more M-carboxylates (M=H, metal, two or more metals, mixtures thereof).

As used herein, the dispersant concentrations are given on an “as delivered” basis. Typically, the active dispersant is delivered with a process oil. The “as delivered” dispersant typically contains from about 20 weight percent to about 80 weight percent, or from about 40 weight percent to about 60 weight percent, of active dispersant in the “as delivered” dispersant product.

### Detergents

Illustrative detergents useful in this disclosure include, for example, alkali metal detergents, alkaline earth metal detergents, or mixtures of one or more alkali metal detergents and one or more alkaline earth metal detergents. A typical detergent is an anionic material that contains a long chain hydrophobic portion of the molecule and a smaller anionic or oleophobic hydrophilic portion of the molecule. The anionic portion of the detergent is typically derived from an organic acid such as a sulfur-containing acid, carboxylic acid (e.g., salicylic acid), phosphorus-containing acid, phenol, or mixtures thereof. The counterion is typically an alkaline earth or alkali metal. The detergent can be overbased as described herein.

The detergent is preferably a metal salt of an organic or inorganic acid, a metal salt of a phenol, or mixtures thereof. The metal is preferably selected from an alkali metal, an alkaline earth metal, and mixtures thereof. The organic or inorganic acid is selected from an aliphatic organic or inorganic acid, a cycloaliphatic organic or inorganic acid, an aromatic organic or inorganic acid, and mixtures thereof.

The metal is preferably selected from an alkali metal, an alkaline earth metal, and mixtures thereof. More preferably, the metal is selected from calcium (Ca), magnesium (Mg), and mixtures thereof.

The organic acid or inorganic acid is preferably selected from a sulfur-containing acid, a carboxylic acid, a phosphorus-containing acid, and mixtures thereof.

Preferably, the metal salt of an organic or inorganic acid or the metal salt of a phenol comprises calcium phenate, calcium sulfonate, calcium salicylate, magnesium phenate, magnesium sulfonate, magnesium salicylate, an overbased detergent, and mixtures thereof.

Salts that contain a substantially stochiometric amount of the metal are described as neutral salts and have a total base number (TBN, as measured by ASTM D2896) of from 0 to 80. Many compositions are overbased, containing large amounts of a metal base that is achieved by reacting an excess of a metal compound (a metal hydroxide or oxide, for example) with an acidic gas (such as carbon dioxide). Useful detergents can be neutral, mildly overbased, or highly overbased. These detergents can be used in mixtures of neutral, overbased, highly overbased calcium salicylate, sulfonates, phenates and/or magnesium salicylate, sulfonates, phenates. The TBN ranges can vary from low, medium to high TBN products, including as low as 0 to as high as 600. Preferably the TBN delivered by the detergent is between 1 and 20. More preferably between 1 and 12. Mixtures of low, medium, high TBN can be used, along with mixtures of calcium and magnesium metal based detergents, and including sulfonates, phenates, salicylates, and carboxylates. A detergent mixture with a metal ratio of 1, in conjunction of a detergent with a metal ratio of 2, and as high as a detergent with a metal ratio of 5, can be used. Borated detergents can also be used.

Alkaline earth phenates are another useful class of detergent. These detergents can be made by reacting alkaline earth metal hydroxide or oxide (CaO, Ca(OH)₂, BaO, Ba(OH)₂, MgO, Mg(OH)₂, for example) with an alkyl phenol or sulfurized alkylphenol. Useful alkyl groups include straight chain or branched C₁-C₃₀ alkyl groups, preferably, C₄-C₂₀ or mixtures thereof. Examples of suitable phenols include isobutylphenol, 2-ethylhexylphenol, nonylphenol, dodecyl phenol, and the like. It should be noted that starting alkylphenols may contain more than one alkyl substituent that are each independently straight chain or branched and can be used from 0.5 to 6 weight percent. When a non-sulfurized alkylphenol is used, the sulfurized product may be obtained by methods well known in the art. These methods include heating a mixture of alkylphenol and sulfurizing agent (including elemental sulfur, sulfur halides such as sulfur dichloride, and the like) and then reacting the sulfurized phenol with an alkaline earth metal base.

In accordance with this disclosure, metal salts of carboxylic acids are preferred detergents. These carboxylic acid detergents may be prepared by reacting a basic metal compound with at least one carboxylic acid and removing free water from the reaction product. These compounds may be overbased to produce the desired TBN level. Detergents made from salicylic acid are one preferred class of detergents derived from carboxylic acids. Useful salicylates include long chain alkyl salicylates. One useful family of compositions is of the formula

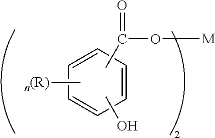

where R is an alkyl group having 1 to about 30 carbon atoms, n is an integer from 1 to 4, and M is an alkaline earth metal. Preferred R groups are alkyl chains of at least C₁₁, preferably C₁₃ or greater. R may be optionally substituted with substituents that do not interfere with the detergent's function. M is preferably, calcium, magnesium, barium, or mixtures thereof. More preferably, M is calcium.

Hydrocarbyl-substituted salicylic acids may be prepared from phenols by the Kolbe reaction (see U.S. Pat. No. 3,595,791). The metal salts of the hydrocarbyl-substituted salicylic acids may be prepared by double decomposition of a metal salt in a polar solvent such as water or alcohol.

Alkaline earth metal phosphates are also used as detergents and are known in the art.

Detergents may be simple detergents or what is known as hybrid or complex detergents. The latter detergents can provide the properties of two detergents without the need to blend separate materials. See U.S. Pat. No. 6,034,039.

Preferred detergents include calcium sulfonates, magnesium sulfonates, calcium salicylates, magnesium salicylates, calcium phenates, magnesium phenates, and other related components (including borated detergents), and mixtures thereof. Preferred mixtures of detergents include magnesium sulfonate and calcium salicylate, magnesium sulfonate and calcium sulfonate, magnesium sulfonate and calcium phenate, calcium phenate and calcium salicylate, calcium phenate and calcium sulfonate, calcium phenate and magnesium salicylate, calcium phenate and magnesium phenate. Overbased detergents are also preferred.

The detergent concentration in the grease compositions of this disclosure can range from about 0.5 to about 6.0 weight percent, preferably about 0.6 to 5.0 weight percent, and more preferably from about 0.8 weight percent to about 4.0 weight percent, based on the total weight of the grease composition.

As used herein, the detergent concentrations are given on an “as delivered” basis. Typically, the active detergent is delivered with a process oil. The “as delivered” detergent typically contains from about 20 weight percent to about 100 weight percent, or from about 40 weight percent to about 60 weight percent, of active detergent in the “as delivered” detergent product.

### Viscosity Modifiers

Viscosity modifiers (also known as viscosity index improvers (VI improvers), and viscosity improvers) can be included in the grease compositions of this disclosure.

Viscosity modifiers provide grease compositions with high and low temperature operability. These additives impart shear stability at elevated temperatures and acceptable viscosity at low temperatures.

Suitable viscosity modifiers include high molecular weight hydrocarbons, polyesters and viscosity modifier dispersants that function as both a viscosity modifier and a dispersant. Typical molecular weights of these polymers are between about 10,000 to 1,500,000, more typically about 20,000 to 1,200,000, and even more typically between about 50,000 and 1,000,000.

Examples of suitable viscosity modifiers are linear or star-shaped polymers and copolymers of methacrylate, butadiene, olefins, or alkylated styrenes. Polyisobutylene is a commonly used viscosity modifier. Another suitable viscosity modifier is polymethacrylate (copolymers of various chain length alkyl methacrylates, for example), some formulations of which also serve as pour point depressants. Other suitable viscosity modifiers include copolymers of ethylene and propylene, hydrogenated block copolymers of styrene and isoprene, and polyacrylates (copolymers of various chain length acrylates, for example). Specific examples include styrene-isoprene or styrene-butadiene based polymers of 50,000 to 200,000 molecular weight.

Olefin copolymers are commercially available from Chevron Oronite Company LLC under the trade designation “PARATONE®” (such as “PARATONE® 8921” and “PARATONE® 8941”); from Afton Chemical Corporation under the trade designation “HiTEC®” (such as “HiTEC® 5850B”; and from The Lubrizol Corporation under the trade designation “Lubrizol® 7067C”. Hydrogenated polyisoprene star polymers are commercially available from Infineum International Limited, e.g., under the trade designation “SV200” and “SV600”. Hydrogenated diene-styrene block copolymers are commercially available from Infineum International Limited, e.g., under the trade designation “SV 50”.

The polymethacrylate or polyacrylate polymers can be linear polymers which are available from Evnoik Industries under the trade designation “Viscoplex®” (e.g., Viscoplex 6-954) or star polymers which are available from Lubrizol Corporation under the trade designation Asteric™ (e.g., Lubrizol 87708 and Lubrizol 87725).

Illustrative vinyl aromatic-containing polymers useful in this disclosure may be derived predominantly from vinyl aromatic hydrocarbon monomer. Illustrative vinyl aromatic-containing copolymers useful in this disclosure may be represented by the following general formula:

A-B

wherein A is a polymeric block derived predominantly from vinyl aromatic hydrocarbon monomer, and B is a polymeric block derived predominantly from conjugated diene monomer.

In an embodiment of this disclosure, the viscosity modifiers may be used in an amount of less than about 10 weight percent, preferably less than about 7 weight percent, more preferably less than about 4 weight percent, and in certain instances, may be used at less than 2 weight percent, preferably less than about 1 weight percent, and more preferably less than about 0.5 weight percent, based on the total weight of the grease composition. Viscosity modifiers are typically added as concentrates, in large amounts of diluent oil.

As used herein, the viscosity modifier concentrations are given on an “as delivered” basis. Typically, the active polymer is delivered with a diluent oil. The “as delivered” viscosity modifier typically contains from 20 weight percent to 75 weight percent of an active polymer for polymethacrylate or polyacrylate polymers, or from 8 weight percent to 20 weight percent of an active polymer for olefin copolymers, hydrogenated polyisoprene star polymers, or hydrogenated diene-styrene block copolymers, in the “as delivered” polymer concentrate.

### Antioxidants

Antioxidants retard the oxidative degradation of base oils during service. Such degradation may result in deposits on metal surfaces, the presence of sludge, or a viscosity increase in the grease compositions. One skilled in the art knows a wide variety of oxidation inhibitors that are useful in grease compositions.

Useful antioxidants include hindered phenols. These phenolic antioxidants may be ashless (metal-free) phenolic compounds or neutral or basic metal salts of certain phenolic compounds. Typical phenolic antioxidant compounds are the hindered phenolics which are the ones which contain a sterically hindered hydroxyl group, and these include those derivatives of dihydroxy aryl compounds in which the hydroxyl groups are in the o- or p-position to each other. Typical phenolic antioxidants include the hindered phenols substituted with C₆+ alkyl groups and the alkylene coupled derivatives of these hindered phenols. Examples of phenolic materials of this type 2-t-butyl-4-heptyl phenol; 2-t-butyl-4-octyl phenol; 2-t-butyl-4-dodecyl phenol; 2,6-di-t-butyl-4-heptyl phenol; 2,6-di-t-butyl-4-dodecyl phenol; 2-methyl-6-t-butyl-4-heptyl phenol; and 2-methyl-6-t-butyl-4-dodecyl phenol. Other useful hindered mono-phenolic antioxidants may include for example hindered 2,6-di-alkyl-phenolic proprionic ester derivatives. Bis-phenolic antioxidants may also be advantageously used in combination with the instant disclosure. Examples of ortho-coupled phenols include: 2,2′-bis(4-heptyl-6-t-butyl-phenol); 2,2′-bis(4-octyl-6-t-butyl-phenol); and 2,2′-bis(4-dodecyl-6-t-butyl-phenol). Para-coupled bisphenols include for example 4,4′-bis(2,6-di-t-butyl phenol) and 4,4′-methylene-bis(2,6-di-t-butyl phenol).

Effective amounts of one or more catalytic antioxidants may also be used. The catalytic antioxidants comprise an effective amount of a) one or more oil soluble polymetal organic compounds; and, effective amounts of b) one or more substituted N,N′-diaryl-o-phenylenediamine compounds or c) one or more hindered phenol compounds; or a combination of both b) and c). Catalytic antioxidants are more fully described in U.S. Pat. No. 8,048,833, herein incorporated by reference in its entirety.

Non-phenolic oxidation inhibitors which may be used include aromatic amine antioxidants and these may be used either as such or in combination with phenolics. Typical examples of non-phenolic antioxidants include: alkylated and non-alkylated aromatic amines such as aromatic monoamines of the formula R⁸R⁹R¹⁰N where R⁸ is an aliphatic, aromatic or substituted aromatic group, R⁹ is an aromatic or a substituted aromatic group, and R¹⁰ is H, alkyl, aryl or R¹¹S(O)_{X}R¹² where R¹¹ is an alkylene, alkenylene, or aralkylene group, R¹² is a higher alkyl group, or an alkenyl, aryl, or alkaryl group, and x is 0, 1 or 2. The aliphatic group R¹² may contain from 1 to about 20 carbon atoms, and preferably contains from about 6 to 12 carbon atoms. The aliphatic group is a saturated aliphatic group. Preferably, both R⁸ and R⁹ are aromatic or substituted aromatic groups, and the aromatic group may be a fused ring aromatic group such as naphthyl. Aromatic groups R⁸ and R⁹ may be joined together with other groups such as S.

Typical aromatic amines antioxidants have alkyl substituent groups of at least about 6 carbon atoms. Examples of aliphatic groups include hexyl, heptyl, octyl, nonyl, and decyl. Generally, the aliphatic groups will not contain more than about 14 carbon atoms. The general types of amine antioxidants useful in the present compositions include diphenylamines, phenyl naphthylamines, phenothiazines, imidodibenzyls and diphenyl phenylene diamines. Mixtures of two or more aromatic amines are also useful. Polymeric amine antioxidants can also be used. Particular examples of aromatic amine antioxidants useful in the present disclosure include: p,p′-dioctyldiphenylamine; t-octylphenyl-alpha-naphthylamine; phenyl-alphanaphthylamine; and p-octylphenyl-alpha-naphthylamine.

Sulfurized alkyl phenols and alkali or alkaline earth metal salts thereof also are useful antioxidants.

Preferred antioxidants include hindered phenols, arylamines. These antioxidants may be used individually by type or in combination with one another. Such additives may be used in an amount of about 0.01 to 5 weight percent, preferably about 0.01 to 1.5 weight percent, more preferably zero to less than 1.5 weight percent, more preferably zero to less than 1 weight percent.

### Pour Point Depressants (PPDs)

Conventional pour point depressants (also known as lube oil flow improvers) may be added to the compositions of the present disclosure if desired. These pour point depressant may be added to grease compositions of the present disclosure to lower the minimum temperature at which the fluid will flow or can be poured. Examples of suitable pour point depressants include polymethacrylates, polyacrylates, polyarylamides, condensation products of haloparaffin waxes and aromatic compounds, vinyl carboxylate polymers, and terpolymers of dialkylfumarates, vinyl esters of fatty acids and allyl vinyl ethers. U.S. Pat. Nos. 1,815,022; 2,015,748; 2,191,498; 2,387,501; 2,655, 479; 2,666,746; 2,721,877; 2,721,878; and 3,250,715 describe useful pour point depressants and/or the preparation thereof. Such additives may be used in an amount of about 0.01 to 5 weight percent, preferably about 0.01 to 1.5 weight percent.

### Seal Compatibility Agents

Seal compatibility agents help to swell elastomeric seals by causing a chemical reaction in the fluid or physical change in the elastomer. Suitable seal compatibility agents for grease compositions include organic phosphates, aromatic esters, aromatic hydrocarbons, esters (butylbenzyl phthalate, for example), and polybutenyl succinic anhydride. Such additives may be used in an amount of about 0.01 to 3 weight percent, preferably about 0.01 to 2 weight percent.

### Antifoam Agents

Antifoam agents may advantageously be added to grease compositions. These agents retard the formation of stable foams. Silicones and organic polymers are typical antifoam agents. For example, polysiloxanes, such as silicon oil or polydimethyl siloxane, provide antifoam properties. Antifoam agents are commercially available and may be used in conventional minor amounts along with other additives such as demulsifiers; usually the amount of these additives combined is less than 1 weight percent and often less than 0.1 weight percent.

### Demulsifiers

A demulsifier may advantageously be added to grease compositions. The demulsifier is used to separate emulsions (e.g., water in oil). An illustrative demulsifying component is described in EP-A-330,522. It is obtained by reacting an alkylene oxide with an adduct obtained by reaction of a bis-epoxide with a polyhydric alcohol. Demulsifiers are commercially available and may be used in conventional minor amounts along with other additives such as antifoam agents; usually the amount of these additives combined is less than 1 weight percent and often less than 0.1 weight percent.

### Inhibitors and Antirust Additives

Antirust additives (or corrosion inhibitors) are additives that protect metal surfaces against chemical attack by water or other contaminants. A wide variety of these are commercially available.

One type of antirust additive is a polar compound that wets the metal surface preferentially, protecting it with a film of oil. Another type of antirust additive absorbs water by incorporating it in a water-in-oil emulsion so that only the oil touches the metal surface. Yet another type of antirust additive chemically adheres to the metal to produce a non-reactive surface. Examples of suitable additives include zinc dithiophosphates, metal phenolates, basic metal sulfonates, fatty acids and amines. Such additives may be used in an amount of about 0.01 to 5 weight percent, preferably about 0.01 to 1.5 weight percent.

### Friction Modifiers

A friction modifier is any material or materials that can alter the coefficient of friction of a surface treated by any grease composition containing such material(s). Friction modifiers, also known as friction reducers, or lubricity agents or oiliness agents, and other such agents that change the ability of base oils, to modify the coefficient of friction of a treated surface may be effectively used in combination with the base oils or grease compositions of the present disclosure if desired. Friction modifiers that lower the coefficient of friction are particularly advantageous in combination with the base oils and lube compositions of this disclosure.

Illustrative friction modifiers may include, for example, organometallic compounds or materials, or mixtures thereof. Illustrative organometallic friction modifiers useful in the grease compositions of this disclosure include, for example, molybdenum amine, molybdenum diamine, an organotungstenate, a molybdenum dithiocarbamate, molybdenum dithiophosphates, molybdenum amine complexes, molybdenum carboxylates, and the like, and mixtures thereof. Similar tungsten based compounds may be preferable.

Other illustrative friction modifiers useful in the grease compositions of this disclosure include, for example, alkoxylated fatty acid esters, alkanolamides, polyol fatty acid esters, borated glycerol fatty acid esters, fatty alcohol ethers, and mixtures thereof.

Illustrative alkoxylated fatty acid esters include, for example, polyoxyethylene stearate, fatty acid polyglycol ester, and the like. These can include polyoxypropylene stearate, polyoxybutylene stearate, polyoxyethylene isosterate, polyoxypropylene isostearate, polyoxyethylene palmitate, and the like.

Illustrative alkanolamides include, for example, lauric acid diethylalkanolamide, palmic acid diethylalkanolamide, and the like. These can include oleic acid diethyalkanolamide, stearic acid diethylalkanolamide, oleic acid diethylalkanolamide, polyethoxylated hydrocarbylamides, polypropoxylated hydrocarbylamides, and the like.

Illustrative polyol fatty acid esters include, for example, glycerol mono-oleate, saturated mono-, di-, and tri-glyceride esters, glycerol mono-stearate, and the like. These can include polyol esters, hydroxyl-containing polyol esters, and the like.

Illustrative borated glycerol fatty acid esters include, for example, borated glycerol mono-oleate, borated saturated mono-, di-, and tri-glyceride esters, borated glycerol mono-sterate, and the like. In addition to glycerol polyols, these can include trimethylolpropane, pentaerythritol, sorbitan, and the like. These esters can be polyol monocarboxylate esters, polyol dicarboxylate esters, and on occasion polyoltricarboxylate esters. Preferred can be the glycerol mono-oleates, glycerol dioleates, glycerol trioleates, glycerol monostearates, glycerol distearates, and glycerol tristearates and the corresponding glycerol monopalmitates, glycerol dipalmitates, and glycerol tripalmitates, and the respective isostearates, linoleates, and the like. On occasion the glycerol esters can be preferred as well as mixtures containing any of these. Ethoxylated, propoxylated, butoxylated fatty acid esters of polyols, especially using glycerol as underlying polyol can be preferred.

Illustrative fatty alcohol ethers include, for example, stearyl ether, myristyl ether, and the like. Alcohols, including those that have carbon numbers from C₃ to C₅₀, can be ethoxylated, propoxylated, or butoxylated to form the corresponding fatty alkyl ethers. The underlying alcohol portion can preferably be stearyl, myristyl, C₁₁-C₁₃ hydrocarbon, oleyl, isosteryl, and the like.

The grease compositions of this disclosure exhibit desired properties in the presence or absence of a friction modifier.

Useful concentrations of friction modifiers may range from 0.01 weight percent to 5 weight percent, or about 0.1 weight percent to about 2.5 weight percent, or about 0.1 weight percent to about 1.5 weight percent, or about 0.1 weight percent to about 1 weight percent. Concentrations of molybdenum-containing materials are often described in terms of Mo metal concentration. Advantageous concentrations of Mo may range from 25 ppm to 700 ppm or more, and often with a preferred range of 50-200 ppm. Friction modifiers of all types may be used alone or in mixtures with the materials of this disclosure. Often mixtures of two or more friction modifiers, or mixtures of friction modifier(s) with alternate surface active material(s), are also desirable.

### Ionic Liquids (ILs)

Ionic liquids are so-called salt melts which are preferably liquid at room temperature and/or by definition have a melting point <100° C. They have almost no vapor pressure and therefore have no cavitation properties. In addition, through the choice of the cations and anions in the ionic liquids, the lifetime and treating effect of the grease composition are increased, and by adjusting the electric conductivity, these liquids can be used in equipment in which there is an electric charge buildup, e.g., electric vehicle powertrains. Suitable cations for ionic liquids include a quaternary ammonium cation, a phosphonium cation, an imidazolium cation, a pyridinium cation, a pyrazolium cation, an oxazolium cation, a pyrrolidinium cation, a piperidinium cation, a thiazolium cation, a guanidinium cation, a morpholinium cation, a trialkylsulfonium cation or a triazolium cation, which may be substituted with an anion selected from the group consisting of [PF₆]⁻, [BF₄]³¹, [CF₃CO₂]³¹, [CF₃SO₃]⁻ as well as its higher homologs, [C₄F₉—SO₃]³¹ or [C₈F₁₇—SO₃]⁻ and higher perfluoroalkylsulfonates, [(CF₃SO₂)₂N]⁻, [(CF₃SO₂)(CF₃COO)N]⁻, [R¹—SO₃]⁻, [R¹—O—SO₃]³¹, [R¹—COO]⁻, Cr⁻, Br⁻, [NO₃]⁻, [N(CN)₂]⁻, [HSO₄]⁻, PF₍₆₋ₓ₎R³ₓ or [R¹R²PO₄]⁻ and the radicals R¹ and R² independently of one another are selected from hydrogen; linear or branched, saturated or unsaturated, aliphatic or alicyclic alkyl groups with 1 to 20 carbon atoms; heteroaryl, heteroaryl-C₁-C₆-alkyl groups with 3 to 8 carbon atoms in the heteroaryl radical and at least one heteroatom of N, O and S, which may be combined with at least one group selected from C₁-C₆ alkyl groups and/or halogen atoms; aryl-aryl C₁-C₆ alkyl groups with 5 to 12 carbon atoms in the aryl radical, which may be substituted with at least one C₁-C₆ alkyl group; R³ may be a perfluoroethyl group or a higher perfluoroalkyl group, x is 1 to 4. However, other combinations are also possible.

Ionic liquids with highly fluorinated anions are especially preferred because they usually have a high thermal stability. The water uptake ability may definitely be reduced by such anions, e.g., in the case of the bis(trifluoromethylsulfonyl)imide anion.

Illustrative ionic liquids include, for example, butylmethylpyrrolidinium bis(trifluoromethylsulfonyl)imide (MBPimide), methylpropylpyrrolidinium bis(trifluoromethylsulfonyl)imide (MPPimide), hexylmethylimidazolium tris(perfluoroethyl)trifluorophosphate (HMIMPFET), hexylmethylimidazolium bis(trifluoromethylsulfonyl)imide (HMIMimide), hexylmethylpyrrolidinium bis(trifluoromethylsulfonyl)imide (HMP), tetrabutylphosphonium tris(perfluoroethyl)trifluorophosphate (BuPPFET), octylmethylimidazolium hexafluorophosphate (OMIM PF6), hexylpyridinium bis(trifluoromethyl)sulfonylimide (Hpyimide), methyltrioctylammonium trifluoroacetate (MOAac), butylmethylpyrrolidinium tris(pentafluoroethyl)trifluorophosphate (MBPPFET), trihexyl(tetradecyl)phosphonium bis(trifluoromethyl sulfonyl)imide (HPDimide), 1-ethyl-3-methylimidazolium ethyl sulfate (EMIM ethyl sulfate), 1-ethyl-3-methylimidazolium bis(trifluoromethyl sulfonyl)imide (EMIMimide), 1-ethyl-2,3-dimethylimidazolium bis(trifluoromethyl sulfonyl)imide (EMMIMimide), N-ethyl-3-methylpyridinium nonafluorobutanesulfonate (EMPyflate), trihexyl(tetradecyl)phosphonium bis(trifluoromethyl sulfonyl)amide, trihexyl(tetradecyl)phosphonium bis(2,4,4-trifluoromethylpentyl)phosphinate, tributyl(tetradecyl)phosphonium dodecylbenzenesulfonate, and the like.

Cation/anion combinations leading to ionic liquids include, for example, dialkylimidazolium, pyridinium, ammonium and phosphonium, etc. with organic anions such as sulfonates, imides, methides, etc., as well as inorganic anions such as halides and phosphates, etc., such that any other combination of cations and anions with which a low melting point can be achieved is also conceivable. Ionic liquids have an extremely low vapor pressure, depending on their chemical structure, are nonflammable and often have thermal stability up to more than 260° C.

The respective desired properties of the grease compositions are achieved with the ionic liquids through a suitable choice of cations and anions. These desirable properties include adjusting electrical conductivity of the grease composition to spread the area of use, increasing the service life and treating effect of the grease composition, and adjusting the viscosity to improve the temperature suitability. Suitable cations for ionic liquids have proven to be a phosphonium cation, an imidazolium cation, a pyridinium cation or a pyrrolidinium cation which may be combined with an anion containing fluorine and selected from bis(trifluoromethylsulfonyl)imide, bis(perfluoroalkylsulfonyl)imide, perfluoroalkyl sulfonate, tris(perfluoroalkyl)methidenes, bis(perfluoroalkyl)imidenes, bis(perfluoroaryl)imides, perfluoroarylperfluoroalkylsulfonylimides and tris(perfluoro-alkyl) trifluorophosphate or with a halogen-free alkyl sulfate anion.

Ionic liquids are preferred with highly fluorinated anions because they usually have a high thermal stability. The water uptake ability may be reduced significantly by such anions, e.g., when using bis(trifluoromethylsulfonyl) anion.

In an embodiment, such ionic liquid additives may be used in an amount of about 0.1 to 10 weight percent, preferably 0.5 to 7.5 weight percent, more preferably about 0.75 to 5 weight percent.

### Extreme Pressure Agents

The lubricating oil compositions can include at least one extreme pressure agent (EP). EP agents that are soluble in the oil include sulphur- and chlorosulphur-containing EP agents, chlorinated hydrocarbon EP agents and phosphorus EP agents. Examples of such EP agents include chlorinated wax; sulphurised olefins (such as sulphurised isobutylene), organic sulphides and polysulphides such as dibenzyldisulphide, bis-(chlorobenzyl)disulphide, dibutyl tetrasulphide, sulphurised methyl ester of oleic acid, sulphurised alkylphenol, sulphurised dipentene, sulphurised terpene, and sulphurised Diels-Alder adducts; phosphosulphurised hydrocarbons such as the reaction product of phosphorus sulphide with turpentine or methyl oleate; phosphorus esters such as the dihydrocarbon and trihydrocarbon phosphites, e.g., dibutyl phosphite, diheptyl phosphite, dicyclohexyl phosphite, pentylphenyl phosphite; dipentylphenyl phosphite, tridecyl phosphite, distearyl phosphite and polypropylene substituted phenol phosphite; metal thiocarbamates such as zinc dioctyldithio carbamate and barium heptylphenol diacid; amine salts of alkyl and dialkylphosphoric acids or derivatives; and mixtures thereof (as described in U.S. Pat. No. 3,197,405).

The extreme pressure agents may be used in an amount of 0.01 to 5 wt %, preferably 0.01 to 1.5 wt %, more preferably 0.01 to 0.2 wt %, still more preferably 0.01 to 0.1 wt % (on an as-received basis) based on the total weight of the lubricating oil composition.

### Metal Passivators

The lubricating oil compositions include at least one metal passivator. The metal passivators/deactivators include, for example, benzotriazole, tolyltriazole, 2-mercaptobenzothiazole, dialkyl-2,5-dimercapto-1,3,4-thiadiazole; N,N′-disalicylideneethylenediamine, N,N′-disalicylidenepropylenediamine; zinc dialkyldithiophosphates and dialkyl dithiocarbamates.

Some embodiments of the disclosure may further comprise a yellow metal passivator. As used herein, “yellow metal” refers to a metallurgical grouping that includes brass and bronze alloys, aluminum bronze, phosphor bronze, copper, copper nickel alloys, and beryllium copper. Typical yellow metal passivators include, for example, benzotriazole, totutriazole, tolyltriazole, mixtures of sodium tolutriazole and tolyltriazole, and combinations thereof. In one particular and non-limiting embodiment, a compound containing tolyltriazole is selected. Typical commercial yellow metal passivators include IRGAMET™-30, and IRGAMET™-42, available from Ciba Specialty Chemicals, now part of BASE, and VANLUBE™ 601 and 704, and CUVAN™ 303 and 484, available from R.T. Vanderbilt Company, Inc.

The metal passivator concentration in the lubricating oils of this disclosure can range from about 0.01 to about 5.0 weight percent, preferably about 0.01 to 3.0 weight percent, and more preferably from about 0.01 weight percent to about 1.5 weight percent, based on the total weight of the lubricating oil.

When grease compositions contain one or more of the additives discussed above, the additive(s) are blended into the composition in an amount sufficient for it to perform its intended function. Typical amounts of such additives useful in the present disclosure are shown in Table 1 below.

It is noted that many of the additives are shipped from the additive manufacturer as a concentrate, containing one or more additives together, with a certain amount of base oil diluents. Accordingly, the weight amounts in the table below, as well as other amounts mentioned herein, are directed to the amount of active ingredient (that is the non-diluent portion of the ingredient). The weight percent (wt %) indicated below is based on the total weight of the grease composition.

TABLE 1
Typical Amounts of Other Grease Components

| | Approximate | Approximate |
|---|---|---|
| Compound | wt % (Useful) | wt % (Preferred) |
| Dispersant | 0.1-20 | 0.1-8 |
| Detergent | 0.1-20 | 0.1-8 |
| Friction Modifier | 0.01-5 | 0.01-1.5 |
| Antioxidant | 0.1-5 | 0.1-1.5 |
| Pour Point Depressant (PPD) | 0.0-5 | 0.01-1.5 |
| Antifoam Agent | 0.001-3 | 0.001-0.15 |
| Demulsifier | 0.001-3 | 0.001-0.15 |
| Viscosity Modifier | 0.1-2 | 0.1-1 |
| (solid polymer basis) | | |
| Antiwear | 0.2-3 | 0.5-1 |
| Inhibitor and Antirust | 0.01-5 | 0.01-1.5 |

The foregoing additives are all commercially available materials. These additives may be added independently but are usually precombined in packages which can be obtained from suppliers of grease additives. Additive packages with a variety of ingredients, proportions and characteristics are available and selection of the appropriate package will take the requisite use of the ultimate composition into account.

### PCT and EP Clauses:

1. A continuous process for preparing a grease thickener product, said process comprising:

(a) introducing a C₃ to C₃₀ hydroxy fatty acid or a C₃ to C₃₀ hydroxy fatty acid ester, and a metal base into a reaction zone;

(b) operating the reaction zone at a temperature between 100° C. and 240° C. to react the C₃ to C₃₀ hydroxy fatty acid or the C₃ to C₃₀ hydroxy fatty acid ester, and the metal base, to produce a grease thickener product;

(c) introducing the grease thickener product of step (b) into a flash chamber zone; and

(d) operating the flash chamber zone at a temperature sufficient to remove volatiles from the grease thickener product of step (b);

wherein continuous process conditions afford a production throughput of grease thickener product of greater than 8,000 pounds per hour.

2. The continuous process of clause 1, further comprising:

(e) passing the grease thickener product of step (b) through a reaction zone back pressure valve operated between 10 and 300 psig;

(f) passing the grease thickener product of step (e) through a heat exchanger operated at a temperature between 100° C. and 240° C.;

(g) passing the grease thickener product of step (f) through a heat exchanger back pressure valve operated between 10 and 300 psig; and

(h) passing the grease thickener product of step (d) through a finishing or shear valve operated between 10 and 300 psig.

3. The continuous process of clause 2, further comprising:

(i) removing and recycling at least a portion of the grease thickener product of step (b) back through the reaction zone; and

(j) passing the grease thickener product of step (i) through a reaction zone recycle pump that is sized to achieve a recycle rate from 0.5 to 20 pounds per minute.

4. The continuous process of clause 3, further comprising:

(k) removing and recycling at least a portion of the grease thickener product of step (d) back through the flash chamber zone; and

(l) passing the grease thickener product of step (k) through a flash chamber zone recycle pump that is sized to achieve a recycle rate from 0.5 to 20 pounds per minute, and a flash chamber zone recycle valve operated between 10 and 300 psig.

5. The continuous process of clause 4, further comprising:

(m) introducing one or more base oils into the reaction zone;

(n) operating the reaction zone at a temperature between 100° C. and 240° C. to react the C₃ to C₃₀ hydroxy fatty acid or the C₃ to C₃₀ hydroxy fatty acid ester, and the metal base, in the presence of the one or more base oils, to produce a grease reaction product.

6. The continuous process of clause 5, further comprising:

(o) introducing one or more grease additives into the grease thickener product of step (d), said one or more grease additives comprising borated esters, polymers, antiwear agents, extreme pressure agents, rust and corrosion inhibitors, boundary lubrication additives, metal passivators, antioxidants, antifoam and defoamants, dispersants, detergents, free radical scavengers, chelating agents, and/or ionic liquids.

7. The continuous process of clause 6, further comprising:

(p) introducing one or more grease additives into the one or more base oils, said one or more grease additives comprising reactive polymers, defoamants, antiwear agents, extreme pressure agents, rust and corrosion inhibitors, boundary lubrication additives, metal passivators, antioxidants, antifoam agents, dispersants, detergents, free radical scavengers, chelating agents, and/or ionic liquids.

8. The continuous process of clause 7, further comprising:

(q) introducing a complexing agent into the grease thickener product of step (e) or step (f).

9. The continuous process of clauses 1-8 wherein the C₃ to C₃₀ hydroxy fatty acid is 12-hydroxy stearic acid, the C₃ to C₃₀ hydroxy fatty acid ester is the methyl or ethyl ester of 12-hydroxy stearic acid, the metal base is lithium oxide, lithium hydroxide, lithium hydroxide monohydrate, or lithium carbonate, the one or more base oils comprise a Group I, Group II, Group III, Group IV, Group V base oil, and combinations thereof, and the complexing agent comprises substituted or unsubstituted dibasic acids, boric acid, methyl salicylate, borated additives, and mixtures thereof.

10. The continuous process of clauses 1-9 wherein continuous process conditions afford a production throughput of grease thickener product of greater than 10,000 pounds per hour.

11. A continuous process for preparing a grease reaction product, said process comprising:

(a) passing one or more base oils through a heat exchanger operated at a temperature between 100° C. and 240° C.;

(b) introducing the one or more base oils of step (a), a C₃ to C₃₀ hydroxy fatty acid or a C₃ to C₃₀ hydroxy fatty acid ester, and a metal base into a reaction zone;

(c) operating the reaction zone at a temperature between 100° C. and 240° C. to react the C₃ to C₃₀ hydroxy fatty acid or the C₃ to C₃₀ hydroxy fatty acid ester, and the metal base, in the presence of the one or more base oils, to produce a grease reaction product;

(d) introducing the grease reaction product of step (c) into a flash chamber zone; and

(e) operating the flash chamber zone at a temperature sufficient to remove volatiles from the grease reaction product of step (c);

wherein continuous process conditions afford a production throughput of grease reaction product of greater than 8,000 pounds per hour.

12. A continuous process for preparing a grease thickener product, said process comprising:

conducting a neutralization reaction comprising reacting a C₃ to C₃₀ hydroxy fatty acid or a C₃ to C₃₀ hydroxy fatty acid ester, and a metal base, under neutralization reaction conditions sufficient to produce a grease thickener product comprising a metal salt of the C₃ to C₃₀ hydroxy fatty acid or the C₃ to C₃₀ hydroxy fatty acid ester;

conducting a nucleation reaction under nucleation reaction conditions sufficient for the metal salt of the C₃ to C₃₀ hydroxy fatty acid or the C₃ to C₃₀ hydroxy fatty acid ester to self-assemble forming a fibrillary network; and

conducting a phase separation under phase separation conditions sufficient to remove byproducts from the grease thickener product, said byproducts comprising methanol and water;

wherein the neutralization reaction is conducted at a temperature above the melting temperature of the C₃ to C₃₀ hydroxy fatty acid or the C₃ to C₃₀ hydroxy fatty acid ester and below the melting temperature of the metal salt of the C₃ to C₃₀ hydroxy fatty acid or the C₃ to C₃₀ hydroxy fatty acid ester; and

wherein, as the metal salt of the C₃ to C₃₀ hydroxy fatty acid or the C₃ to C₃₀ hydroxy fatty acid ester is produced in the neutralization reaction, the difference between the melting temperature of the metal salt of the C₃ to C₃₀ hydroxy fatty acid or the C₃ to C₃₀ hydroxy fatty acid ester and the melting temperature of the C₃ to C₃₀ hydroxy fatty acid or the C₃ to C₃₀ hydroxy fatty acid ester creates a chemical driving force that initiates the nucleation reaction forming the fibrillary network.

13. The continuous process of clause 12 wherein the C₃ to C₃₀ hydroxy fatty acid or a C₃ to C₃₀ hydroxy fatty acid ester comprises methyl-12-hydroxystearate, the metal base comprises lithium hydroxide, and the metal salt of the C₃ to C₃₀ hydroxy fatty acid or the C₃ to C₃₀ hydroxy fatty acid ester comprises lithium 12-hydroxystearate.

14. A continuous process for preparing a grease reaction product, said process comprising:

conducting a neutralization reaction comprising reacting a C₃ to C₃₀ hydroxy fatty acid or a C₃ to C₃₀ hydroxy fatty acid ester, and a metal base, in the presence of one or more base oils, under neutralization reaction conditions sufficient to produce a grease reaction product comprising a metal salt of the C₃ to C₃₀ hydroxy fatty acid or the C₃ to C₃₀ hydroxy fatty acid ester;

conducting a nucleation reaction under nucleation reaction conditions sufficient for the metal salt of the C₃ to C₃₀ hydroxy fatty acid or the C₃ to C₃₀ hydroxy fatty acid ester to self-assemble forming a fibrillary network; and

conducting a phase separation under phase separation conditions sufficient to remove byproducts from the grease reaction product, said byproducts comprising methanol and water;

wherein the neutralization reaction is conducted at a temperature above the melting temperature of the C₃ to C₃₀ hydroxy fatty acid or the C₃ to C₃₀ hydroxy fatty acid ester and below the melting temperature of the metal salt of the C₃ to C₃₀ hydroxy fatty acid or the C₃ to C₃₀ hydroxy fatty acid ester; and

wherein, as the metal salt of the C₃ to C₃₀ hydroxy fatty acid or the C₃ to C₃₀ hydroxy fatty acid ester is produced in the neutralization reaction, the difference between the melting temperature of the metal salt of the C₃ to C₃₀ hydroxy fatty acid or the C₃ to C₃₀ hydroxy fatty acid ester and the melting temperature of the C₃ to C₃₀ hydroxy fatty acid or the C₃ to C₃₀ hydroxy fatty acid ester creates a chemical driving force that initiates the nucleation reaction forming the fibrillary network.

15. The continuous process of clause 14 wherein continuous process conditions afford a production throughput of grease reaction product of greater than 8,000 pounds per hour.

All patents and patent applications, test procedures (such as ASTM methods, UL methods, and the like), and other documents cited herein are fully incorporated by reference to the extent such disclosure is not inconsistent with this disclosure and for all jurisdictions in which such incorporation is permitted.

When numerical lower limits and numerical upper limits are listed herein, ranges from any lower limit to any upper limit are contemplated. While the illustrative embodiments of the disclosure have been described with particularity, it will be understood that various other modifications will be apparent to and can be readily made by those skilled in the art without departing from the spirit and scope of the disclosure. Accordingly, it is not intended that the scope of the claims appended hereto be limited to the examples and descriptions set forth herein but rather that the claims be construed as encompassing all the features of patentable novelty which reside in the present disclosure, including all features which would be treated as equivalents thereof by those skilled in the art to which the disclosure pertains.

The present disclosure has been described above with reference to numerous embodiments and specific examples. Many variations will suggest themselves to those skilled in this art in light of the above detailed description. All such obvious variations are within the full intended scope of the appended claims.

1. A continuous process for preparing a grease thickener product, said process comprising:
(a) introducing a C₃ to C₃₀ hydroxy fatty acid or a C₃ to C₃₀ hydroxy fatty acid ester, and a metal base into a reaction zone;
(b) operating the reaction zone at a temperature between about 100° C. and about 240° C. to react the C₃ to C₃₀ hydroxy fatty acid or the C₃ to C₃₀ hydroxy fatty acid ester, and the metal base, to produce a grease thickener product;
(c) introducing the grease thickener product of step (b) into a flash chamber zone; and
(d) operating the flash chamber zone at a temperature sufficient to remove volatiles from the grease thickener product of step (b);
wherein continuous process conditions afford a production throughput of grease thickener product of greater than about 8,000 pounds per hour.

2. The continuous process of claim 1, further comprising:
(e) passing the grease thickener product of step (b) through a reaction zone back pressure valve operated between about 10 and about 300 psig;
(f) passing the grease thickener product of step (e) through a heat exchanger operated at a temperature between about 100° C. and 240° C.;
(g) passing the grease thickener product of step (f) through a heat exchanger back pressure valve operated between about 10 and about 300 psig; and
(h) passing the grease thickener product of step (d) through a finishing or shear valve operated between about 10 and about 300 psig.

3. The continuous process of claim 1 further comprising:
(i) removing and recycling at least a portion of the grease thickener product of step (b) back through the reaction zone; and
(j) passing the grease thickener product of step (i) through a reaction zone recycle pump that is sized to achieve a recycle rate from about 0.5 to about 20 pounds per minute.

4. The continuous process of claim 1 further comprising:
(k) removing and recycling at least a portion of the grease thickener product of step (d) back through the flash chamber zone; and
(l) passing the grease thickener product of step (k) through a flash chamber zone recycle pump that is sized to achieve a recycle rate from about 0.5 to about 20 pounds per minute, and a flash chamber zone recycle valve operated between about 10 and about 300 psig.

5. The continuous process of claim 1 further comprising:
(m)introducing one or more base oils into the reaction zone;
(n) operating the reaction zone at a temperature between about 100° C. and about 240° C. to react the C₃ to C₃₀ hydroxy fatty acid or the C₃ to C₃₀ hydroxy fatty acid ester, and the metal base, in the presence of the one or more base oils, to produce a grease reaction product.

6. The continuous process of claim 1 further comprising:
(o) introducing one or more grease additives into the grease thickener product of step (d), said one or more grease additives comprising borated esters, polymers, antiwear agents, extreme pressure agents, rust and corrosion inhibitors, boundary lubrication additives, metal passivators, antioxidants, antifoam and defoamants, dispersants, detergents, free radical scavengers, chelating agents, and/or ionic liquids.

7. The continuous process of claim 5 further comprising:
(p) introducing one or more grease additives into the one or more base oils, said one or more grease additives comprising reactive polymers, defoamants, antiwear agents, extreme pressure agents, rust and corrosion inhibitors, boundary lubrication additives, metal passivators, antioxidants, antifoam agents, dispersants, detergents, free radical scavengers, chelating agents, and/or ionic liquids.

8. The continuous process of claim 1 further comprising:
(q) introducing a complexing agent into the grease thickener product of step (e) or step (f).

9. The continuous process of claim 1 wherein the C₃ to C₃₀ hydroxy fatty acid is 12-hydroxy stearic acid.

10. The continuous process of claim 1 wherein the C₃ to C₃₀ hydroxy fatty acid ester is the methyl or ethyl ester of 12-hydroxy stearic acid.

11. The continuous process of claim 1 wherein the metal base is lithium oxide, lithium hydroxide, lithium hydroxide monohydrate, or lithium carbonate.

12. The continuous process of claim 5 wherein the one or more base oils comprise a Group I, Group II, Group III, Group IV, Group V base oil, and combinations thereof.

13. The continuous process of claim 8 wherein the complexing agent comprises substituted or unsubstituted dibasic acids, boric acid, methyl salicylate, borated additives, and mixtures thereof.

14. The continuous process of claim 1 wherein continuous process conditions afford a production throughput of grease thickener product of greater than about 10,000 pounds per hour.

15. The continuous process of claim 1 wherein continuous process conditions afford a production throughput of grease thickener product of greater than about 12,000 pounds per hour.

16. A continuous process for preparing a grease reaction product, said process comprising:
(a) passing one or more base oils through a heat exchanger operated at a temperature between about 100° C. and 240° C.;
(b) introducing the one or more base oils of step (a), a C₃ to C₃₀ hydroxy fatty acid or a C₃ to C₃₀ hydroxy fatty acid ester, and a metal base into a reaction zone;
(c) operating the reaction zone at a temperature between about 100° C. and about 240° C. to react the C₃ to C₃₀ hydroxy fatty acid or the C₃ to C₃₀ hydroxy fatty acid ester, and the metal base, in the presence of the one or more base oils, to produce a grease reaction product;
(d) introducing the grease reaction product of step (c) into a flash chamber zone; and
(e) operating the flash chamber zone at a temperature sufficient to remove volatiles from the grease reaction product of step (c);
wherein continuous process conditions afford a production throughput of grease reaction product of greater than about 8,000 pounds per hour.

17. The continuous process of claim 16, further comprising:
(f) passing the grease reaction product of step (c) through a reaction zone back pressure valve operated between about 10 and about 300 psig;
(g) passing the grease reaction product of step (f) through a heat exchanger operated at a temperature between about 100° C. and 240° C.;
(h) passing the grease reaction product of step (g) through a heat exchanger back pressure valve operated between about 10 and about 300 psig; and
(i) passing the grease reaction product of step (e) through a finishing or shear valve operated between about 10 and about 300 psig.

18. The continuous process of claim 16 further comprising:
(j) removing and recycling at least a portion of the grease reaction product of step (c) back through the reaction zone; and
(k) passing the grease reaction product of step (j) through a reaction zone recycle pump that is sized to achieve a recycle rate from about 0.5 to about 20 pounds per minute.

19. The continuous process of claim 16 further comprising:
(l) removing and recycling at least a portion of the grease reaction product of step (e) back through the flash chamber zone; and
(m)passing the grease reaction product of step (l) through a flash chamber zone recycle pump that is sized to achieve a recycle rate from about 0.5 to about 20 pounds per minute, and a flash chamber zone recycle valve operated between about 10 and about 300 psig.

20. The continuous process of claim 16 further comprising:
(n) introducing one or more grease additives into the grease reaction product of step (e), said one or more grease additives comprising borated esters, polymers, antiwear agents, extreme pressure agents, rust and corrosion inhibitors, boundary lubrication additives, metal passivators, antioxidants, antifoam and defoamants, dispersants, detergents, free radical scavengers, chelating agents, and/or ionic liquids.

21. The continuous process of claim 16 further comprising:
(o) introducing one or more grease additives into the one or more base oils, said one or more grease additives comprising reactive polymers, defoamants, antiwear agents, extreme pressure agents, rust and corrosion inhibitors, boundary lubrication additives, metal passivators, antioxidants, antifoam agents, dispersants, detergents, free radical scavengers, chelating agents, and/or ionic liquids.

22. The continuous process of claim 16 further comprising:
(p) introducing a complexing agent into the grease reaction product of step (f) or step (g).

23. The continuous process of claim 16 wherein the C₃ to C₃₀ hydroxy fatty acid is 12-hydroxy stearic acid.

24. The continuous process of claim 16 wherein the C₃ to C₃₀ hydroxy fatty acid ester is the methyl or ethyl ester of 12-hydroxy stearic acid.

25. The continuous process of claim 16 wherein the metal base is lithium oxide, lithium hydroxide, lithium hydroxide monohydrate, or lithium carbonate.

26. The continuous process of claim 16 wherein the one or more base oils comprise a Group I, Group II, Group III, Group IV, Group V base oil, and combinations thereof.

27. The continuous process of claim 22 wherein the complexing agent comprises substituted or unsubstituted dibasic acids, boric acid, methyl salicylate, borated additives, and mixtures thereof.

28. The continuous process of claim 16 wherein continuous process conditions afford a production throughput of grease reaction product of greater than about 10,000 pounds per hour.

29. The continuous process of claim 16 wherein continuous process conditions afford a production throughput of grease reaction product of greater than about 12,000 pounds per hour.

30. A continuous process for preparing a grease thickener product, said process comprising:
conducting a neutralization reaction comprising reacting a C₃ to C₃₀ hydroxy fatty acid or a C₃ to C₃₀ hydroxy fatty acid ester, and a metal base, under neutralization reaction conditions sufficient to produce a grease thickener product comprising a metal salt of the C₃ to C₃₀ hydroxy fatty acid or the C₃ to C₃₀ hydroxy fatty acid ester;
conducting a nucleation reaction under nucleation reaction conditions sufficient for the metal salt of the C₃ to C₃₀ hydroxy fatty acid or the C₃ to C₃₀ hydroxy fatty acid ester to self-assemble forming a fibrillary network; and
conducting a phase separation under phase separation conditions sufficient to remove byproducts from the grease thickener product, said byproducts comprising methanol and water;
wherein the neutralization reaction is conducted at a temperature above the melting temperature of the C₃ to C₃₀ hydroxy fatty acid or the C₃ to C₃₀ hydroxy fatty acid ester and below the melting temperature of the metal salt of the C₃ to C₃₀ hydroxy fatty acid or the C₃ to C₃₀ hydroxy fatty acid ester; and
wherein, as the metal salt of the C₃ to C₃₀ hydroxy fatty acid or the C₃ to C₃₀ hydroxy fatty acid ester is produced in the neutralization reaction, the difference between the melting temperature of the metal salt of the C₃ to C₃₀ hydroxy fatty acid or the C₃ to C₃₀ hydroxy fatty acid ester and the melting temperature of the C₃ to C₃₀ hydroxy fatty acid or the C₃ to C₃₀ hydroxy fatty acid ester creates a chemical driving force that initiates the nucleation reaction forming the fibrillary network.

31. The continuous process of claim 30 wherein continuous process conditions afford a production throughput of grease thickener product of greater than about 8,000 pounds per hour.

32. The continuous process of claim 30 wherein the C₃ to C₃₀ hydroxy fatty acid or a C₃ to C₃₀ hydroxy fatty acid ester comprises methyl-12-hydroxystearate and the metal base comprises lithium hydroxide.

33. The continuous process of claim 30 wherein the metal salt of the C₃ to C₃₀ hydroxy fatty acid or the C₃ to C₃₀ hydroxy fatty acid ester comprises lithium 12-hydroxystearate.

34. A continuous process for preparing a grease reaction product, said process comprising:
conducting a neutralization reaction comprising reacting a C₃ to C₃₀ hydroxy fatty acid or a C₃ to C₃₀ hydroxy fatty acid ester, and a metal base, in the presence of one or more base oils, under neutralization reaction conditions sufficient to produce a grease reaction product comprising a metal salt of the C₃ to C₃₀ hydroxy fatty acid or the C₃ to C₃₀ hydroxy fatty acid ester;
conducting a nucleation reaction under nucleation reaction conditions sufficient for the metal salt of the C₃ to C₃₀ hydroxy fatty acid or the C₃ to C₃₀ hydroxy fatty acid ester to self-assemble forming a fibrillary network; and
conducting a phase separation under phase separation conditions sufficient to remove byproducts from the grease reaction product, said byproducts comprising methanol and water;
wherein the neutralization reaction is conducted at a temperature above the melting temperature of the C₃ to C₃₀ hydroxy fatty acid or the C₃ to C₃₀ hydroxy fatty acid ester and below the melting temperature of the metal salt of the C₃ to C₃₀ hydroxy fatty acid or the C₃ to C₃₀ hydroxy fatty acid ester; and
wherein, as the metal salt of the C₃ to C₃₀ hydroxy fatty acid or the C₃ to C₃₀ hydroxy fatty acid ester is produced in the neutralization reaction, the difference between the melting temperature of the metal salt of the C₃ to C₃₀ hydroxy fatty acid or the C₃ to C₃₀ hydroxy fatty acid ester and the melting temperature of the C₃ to C₃₀ hydroxy fatty acid or the C₃ to C₃₀ hydroxy fatty acid ester creates a chemical driving force that initiates the nucleation reaction forming the fibrillary network.

35. The continuous process of claim 34 wherein continuous process conditions afford a production throughput of grease reaction product of greater than about 8,000 pounds per hour.

36. The continuous process of claim 34 wherein the C₃ to C₃₀ hydroxy fatty acid or a C₃ to C₃₀ hydroxy fatty acid ester comprises methyl-12-hydroxystearate and the metal base comprises lithium hydroxide.

37. The continuous process of claim 34 wherein the metal salt of the C₃ to C₃₀ hydroxy fatty acid or the C₃ to C₃₀ hydroxy fatty acid ester comprises lithium 12-hydroxystearate.

38. A grease thickener product prepared by the continuous process of claim 1.

39. A grease reaction product prepared by the continuous process of claim 16.

40. A grease thickener product prepared by the continuous process of claim 30.

41. A grease reaction product prepared by the continuous process of claim 34.
